(19) United States
(12) Patent Application Publication
(10) Pub. No.: US 2025/0319315 A1
(43) Pub. Date: Oct. 16, 2025

(54) NEUROSTIMULATION TESTING WITH VARIABLE RAMP

(71) Applicant: Boston Scientific Neuromodulation Corporation, Valencia, CA (US)

(72) Inventors: Lisa Denise Moore, Glendale, CA (US); Rafael Carbunaru, Valley Village, CA (US)

(21) Appl. No.: 19/171,721

(22) Filed: Apr. 7, 2025

Related U.S. Application Data
(60) Provisional application No. 63/632,947, filed on Apr. 11, 2024.

(51) Int. Cl. A61N 1/372; A61N 1/36; G16H 10/60; G16H 20/40; G16H 50/70

(52) U.S. Cl. CPC A61N 1/37241; A61N 1/3615; A61N 1/36171; A61N 1/36175; A61N 1/37247; G16H 10/60; G16H 20/40; G16H 50/70

(57) ABSTRACT

A system may include a neurostimulator and a processing system. The neurostimulator may be configured to deliver electrical energy according to a stimulation parameter set. The stimulation parameter set may include at least one adjustable parameter. The processing system may be configured to perform a testing process to test delivering electrical energy by automatically progressing through a sequence of values for the at least one adjustable parameter in the parameter set. A first value and a second value in the sequence of values are separated by an initial parameter change interval and a second to last value and the last value in the sequence of values are separated by a final parameter change interval. Progressing through the sequence of values includes reducing the parameter change interval from the initial parameter change interval to the final parameter change interval.

### CLAIM OF PRIORITY

This application claims the benefit of U.S. Provisional Application No. 63/632,947, filed on Apr. 11, 2024, which is hereby incorporated by reference in its entirety.

### TECHNICAL FIELD

This document relates generally to medical devices, and more particularly, to systems, devices and methods for testing stimulation parameters to program a neurostimulation system.

### BACKGROUND

Medical devices may include therapy-delivery devices configured to deliver a therapy to a patient and/or monitors configured to monitor a patient condition via user input and/or sensor(s). Examples include wearable devices such as but not limited to, transcutaneous electrical neural stimulators (TENS), external or implantable stimulation devices such as but not limited to spinal cord stimulators (SCS) to treat chronic pain, cortical and Deep Brain Stimulators (DBS) to treat motor and psychological disorders, Peripheral Nerve Stimulation (PNS), Functional Electrical Stimulation (FES), and other neural stimulators to treat urinary incontinence, sleep apnea, shoulder subluxation, and the like.

A therapy device may be configured or programmed to treat a condition. Thus, by way of example and not limitation, a DBS system may be configured to treat motor disorders such as, but not limited to, tremor, bradykinesia, and dyskinesia associated with Parkinson's Disease (PD). In another nonlimiting example, a stimulation device, such as neurostimulation device (e.g., DBS, SCS, PNS or TENS), may be configured to treat pain. Settings of the therapy device, including stimulation parameters, may be programmed based on observed clinical effects so that the therapy provides desirable intended effects (e.g., reduced tremor, bradykinesia, and dyskinesia for a PD therapy, desirable pain relief or paresthesia coverage for a pain therapy) while avoiding undesirable side effects.

A programming process may be used to test different stimulation parameters as part of a process to program the therapy device to provide an appropriate therapy for the patient. This disclosure provides an improved system and process for programming the therapy device.

### SUMMARY

An example (e.g., “Example 1”) of a system may include a neurostimulator and a processing system. The neurostimulator may be configured to deliver electrical energy according to a stimulation parameter set. The stimulation parameter set may include at least one adjustable parameter. The processing system may be configured to perform a testing process to test delivering electrical energy by automatically progressing through a sequence of values for the at least one adjustable parameter in the parameter set. A first value and a second value in the sequence of values are separated by an initial parameter change interval and a second to last value and the last value in the sequence of values are separated by a final parameter change interval. Progressing through the sequence of values includes reducing the parameter change interval from the initial parameter change interval to the final parameter change interval.

In Example 2, the subject matter of Example 1 may optionally be configured such that the at least one adjustable parameter includes at least one of: an amplitude; a pulse width; a pulse frequency; fractionalization values for at least two electrodes; or a composite parameter. The composite parameter may provide an indicator of delivered charge or an indicator of delivered dose based at least in part on values for at least two of an amplitude, a pulse width or a pulse frequency.

In Example 3, the subject matter of any one or more of Examples 1-2 may optionally be configured such that automatically progressing through the sequence of values includes using a same parameter change interval to progress through at least two consecutive values in the sequence of values and subsequently reducing the parameter change interval before progressing through a subsequent value in the sequence of values.

In Example 4, the subject matter of any one or more of Examples 1-3 may optionally be configured such that automatically progressing through the sequence of values includes using at least three different parameter change intervals to progress through three consecutive values in the sequence of values.

In Example 5, the subject matter of any one or more of Examples 1-4 may optionally be configured such that timing for delivering electrical energy using a current one of the sequence of values before moving to a subsequent one of the sequence of values is longer than a wash-in time for the electrical energy to cause a neural stimulation effect.

In Example 6, the subject matter of any one or more of Examples 1-5 may optionally be configured such that the testing process is pre-programmed to test a specific sequence of values with the parameter change interval progressively reduced from the initial parameter change interval to the final parameter change interval.

In Example 7, the subject matter of any one or more of Examples 1-5 may optionally be configured to further include receiving at least one input. The parameter change interval may depend on the at least one received input.

In Example 8, the subject matter of Example 7 may optionally be configured such that the at least one input includes data indicative of a spatial relationship between a representation of an anatomical structure and a stimulation field model representing a volume of tissue activated by the delivered electrical energy. The parameter change interval may depend on the spatial relationship.

In Example 9, the subject matter of any one or more of Examples 7-8 may optionally be configured such that the at least one input includes recorded patient data of a stimulation effect for a specific stimulation parameter set. The testing process may include estimating a stimulation effect probability for other stimulation parameter sets using the recorded patient data, and the parameter change interval may depend on the estimated stimulation effect probability.

In Example 10, the subject matter of any one or more of Examples 7-9 may optionally be configured such that the at least one input includes user data indicative of a user's programming preference. The parameter change interval may depend on the user's programming preference.

In Example 11, the subject matter of any one or more of Examples 7-10 may optionally be configured such that the at least one input includes aggregate data collected from multiple patients. The testing process may include estimating a stimulation effect probability for other stimulation parameter sets using the aggregate data, and the parameter change interval may depend on the estimated stimulation effect probability.

In Example 12, the subject matter of any one or more of Examples 7-11 may optionally be configured such that the at least one input includes an estimated change in stimulation effect for the delivered electrical energy between adjacent steps in the sequence of values. The parameter change interval may depend on the estimated change.

In Example 13, the subject matter of any one or more of Examples 7-12 may optionally be configured such that the at least one input includes a manual input indicative of at least one transition point in the sequence of values for transitioning to a different parameter change interval. The parameter change interval may depend on the at least one transition point.

In Example 14, the subject matter of any one or more of Examples 1-13 may optionally be configured to further include receiving a sensitive patient indicator, and the parameter change interval depends on the sensitive patient indicator.

In Example 15, the subject matter of any one or more of Examples 1-14 may optionally be configured to further include receiving a user-inputted override for overriding the processing system from progressively reducing the parameter change interval when automatically progressing through the sequence of values for the at least one adjustable parameter in the parameter set.

Example 16 includes subject matter (such as a method, means for performing acts, machine readable medium including instructions that when performed by a machine cause the machine to perform acts, or an apparatus to perform). The subject matter may include delivering electrical energy according to a stimulation parameter set using a neurostimulator. The stimulation parameter set may include at least one adjustable parameter. The subject matter may further include using a processing system to perform a testing process to test delivering electrical energy by automatically progressing through a sequence of values for the at least one adjustable parameter in the parameter set. A first value and a second value in the sequence of values may be separated by an initial parameter change interval. A second to last value and the last value in the sequence of values may be separated by a final parameter change interval. Progressing through the sequence of values may include reducing the parameter change interval from the initial parameter change interval to the final parameter change interval.

In Example 17, the subject matter of Example 16 may optionally be configured such that the at least one adjustable parameter includes at least one of: an amplitude; a pulse width; a pulse frequency; fractionalization values for at least two electrodes; or a composite parameter. The composite parameter may provide an indicator of delivered charge or an indicator of delivered dose based at least in part on values for at least two of an amplitude, a pulse width or a pulse frequency.

In Example 18, the subject matter of any one or more of Examples 16-17 may optionally be configured such that automatically progressing through the sequence of values includes using a same parameter change interval to progress through at least two consecutive values in the sequence of values and subsequently reducing the parameter change interval before progressing through a subsequent value in the sequence of values; and/or using at least three different parameter change intervals to progress through three consecutive values in the sequence of values.

In Example 19, the subject matter of any one or more of Examples 16-18 may optionally be configured such that timing for delivering electrical energy using a current one of the sequence of values before moving to a subsequent one of the sequence of values is longer than a wash-in time for the electrical energy to cause a neural stimulation effect.

In Example 20, the subject matter of any one or more of Examples 16-19 may optionally be configured such that the testing process is pre-programmed to test a specific sequence of values with the parameter change interval progressively reduced from the initial parameter change interval to the final parameter change interval.

In Example 21, the subject matter of any one or more of Examples 16-19 may optionally be configured to further include receiving at least one input. The parameter change interval may depend on the at least one received input.

In Example 22, the subject matter of Example 21 may optionally be configured such that the at least one input includes data indicative of a spatial relationship between a representation of an anatomical structure and a stimulation field model representing a volume of tissue activated by the delivered electrical energy. The parameter change interval may depend on the spatial relationship.

In Example 23, the subject matter of any one or more of Examples 21-22 may optionally be configured such that the at least one input includes recorded patient data of a stimulation effect for a specific stimulation parameter set. The method may include estimating a stimulation effect probability for other stimulation parameter sets using the recorded patient data, and the parameter change interval may depend on the estimated stimulation effect probability.

In Example 24, the subject matter of any one or more of Examples 21-23 may optionally be configured such that the at least one input includes user data indicative of a user's programming preference. The parameter change interval may depend on the user's programming preference.

In Example 25, the subject matter of any one or more of Examples 21-24 may optionally be configured such that the at least one input includes aggregate data collected from multiple patients. The method may include estimating a stimulation effect probability for other stimulation parameter sets using the aggregate data, and the parameter change interval may depend on the estimated stimulation effect probability.

In Example 26, the subject matter of any one or more of Examples 21-25 may optionally be configured such that the at least one input includes an estimated change in stimulation effect for the delivered electrical energy between adjacent steps in the sequence of values. The parameter change interval may depend on the estimated change.

In Example 27, the subject matter of any one or more of Examples 21-26 may optionally be configured such that the at least one input includes a manual input indicative of at least one transition point in the sequence of values for transitioning to a different parameter change interval. The parameter change interval may depend on the at least one transition point.

In Example 28, the subject matter any one or more of claims 16-20 may optionally be configured such that the at least one input includes at least two inputs selected from: data indicative of a spatial relationship between a representation of an anatomical structure and a stimulation field model representing a volume of tissue activated by the delivered electrical energy; recorded patient data of a stimulation effect for a specific stimulation parameter set, and the method includes estimating a stimulation effect probability for other stimulation parameter sets using the recorded patient data; user data indicative of a user's programming preference; aggregate data collected from multiple patients, and the method includes estimating a stimulation effect probability for other stimulation parameter sets using the aggregate data; an estimated change in stimulation effect for the delivered electrical energy between adjacent steps in the sequence of values; or a manual input indicative of at least one transition point in the sequence of values for transitioning to a different parameter change interval. The parameter change interval may depend on the at least two inputs.

In Example 29, the subject matter of any one or more of Examples 16-28 may optionally be configured to further include receiving a sensitive patient indicator, and the parameter change interval depends on the sensitive patient indicator.

In Example 30, the subject matter of any one or more of Examples 16-29 may optionally be configured to further include receiving a user-inputted override for overriding the processing system from progressively reducing the parameter change interval when automatically progressing through the sequence of values for the at least one adjustable parameter in the parameter set.

Example 31 includes subject matter that includes non-transitory machine-readable medium including instructions, which when executed by a machine, cause the machine to perform a method. The method may include, by way of example and not limitation, any of the subject matter for one or more of Examples 16-30. The machine-readable medium may include instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like. The term “machine-readable medium” is intended to include at least one machine-readable medium (e.g., two or more media which may be of the same type of media (such as but not limited to different nonvolatile semiconductor memory arrays) or different type of media (such as but not limited to a hard disk and a non-volatile semiconductor memory array). Furthermore, the term “machine” may include at least one processor, including one processor to implement all of the instructions, at least two processors where one processor operates on some of the instructions and other processor(s) operate on other instructions, or at least two processors where each processor is capable of operating on the same instructions. Thus, for example, distributed systems or systems with shared resources are contemplated.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various examples are illustrated by way of example in the figures of the accompanying drawings. Such examples are demonstrative and not intended to be exhaustive or exclusive examples of the present subject matter.

FIG. 1 illustrates an example of an electrical stimulation system that may be used to deliver deep brain stimulation (DBS).

FIG. 2 illustrates an example of an implantable pulse generator (IPG) that may be used in a DBS system.

FIGS. 3A-3B illustrate examples of leads that may be coupled to an IPG to deliver electrostimulation such as DBS.

FIG. 4 illustrates an example of a computing device for programming or controlling the operation of an electrostimulation system.

FIG. 5 illustrates an example of a stimulation parameter control system and a part of the environment in which it may operate.

FIG. 6 illustrates, by way of example, an example of an electrical therapy-delivery system.

FIG. 7 illustrates, by way of example and not limitation, an implantable electrical therapy-delivery system.

FIG. 8 illustrates a therapy being delivered according to a parameter set.

FIG. 9 illustrates a therapy space, which includes different parameter sets potentially available for delivering the therapy.

FIGS. 10A-10B illustrate, by way of example and not limitation, the use of static ramping protocols for two patients who experience some stimulation effects differently.

FIGS. 11A-11B illustrate, by way of example and not limitation, the use of a variable ramping protocols for two patients who experience some stimulation effects differently.

FIG. 12 illustrates an example of a ramping protocol for testing different parameter values in a parameter set. Each dot may represent a tested parameter value.

FIG. 13 illustrates, by way of example and not limitation, a ramping protocol.

FIG. 14 illustrates, by way of example and not limitation, a method for testing parameter values for an adjustable parameter in a parameter set.

FIG. 15 illustrates, by way of example and not limitation, a testing process.

FIG. 16 illustrates, by way of example and not limitation, a more detailed example of a testing process that includes determining a dynamic parameter change increment used to adjust a value of the adjustable parameter in the parameter set.

FIG. 17 illustrates, by way of example and not limitation, a system for providing a variable parameter value interval to test parameter values within a stimulation parameter set.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

This disclosure proposes a more intelligent programming process to test different stimulation parameters. The programming process may include variable ramping of stimulation parameter values (e.g., amplitude). Variable amplitude ramping allows for faster or larger amplitude increases under established safer conditions to a set threshold, and slower or smaller amplitude increases beyond that threshold in regions where large/rapid stimulation changes can induce significant patient discomfort. For example, instead of a static ramp that increases stimulation amplitude uniformly to the new target, the speed for increasing stimulation amplitude may be accelerated in regions where stimulation change is largely imperceptible to the patient (or does not cause discomfort) and then the speed for increasing stimulation amplitude may be slowed after stimulation becomes perceptible and is estimated to be near a level of discomfort. The ramping may accommodate thresholds different than the perception and discomfort thresholds.

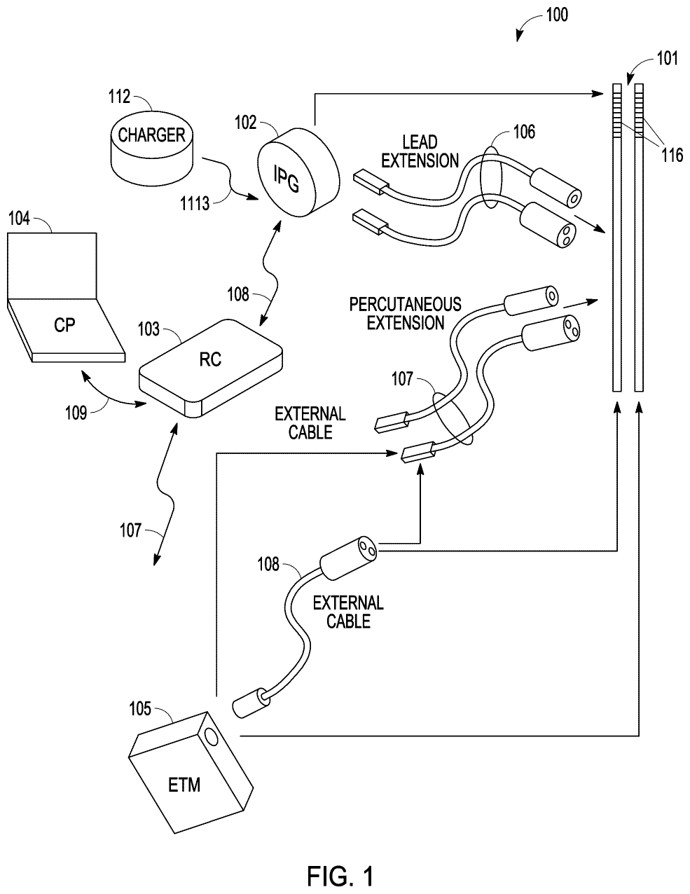

FIG. 1 illustrates, by way of example and not limitation, an electrical stimulation system 100, which may be used to deliver DBS. The electrical stimulation system 100 may generally include a one or more (illustrated as two) of implantable neurostimulation leads 101, a waveform generator such as an implantable pulse generator (IPG) 102, an external remote controller (RC) 103, a clinician programmer (CP) 104, and an external trial modulator (ETM) 105. The IPG 102 may be physically connected via one or more percutaneous lead extensions 106 to the neurostimulation lead(s) 101, which carry a plurality of electrodes 116. The electrodes, when implanted in a patient, form an electrode arrangement. As illustrated, the neurostimulation leads 101 may be percutaneous leads with the electrodes arranged in-line along the neurostimulation leads or about a circumference of the neurostimulation leads. Any suitable number of neurostimulation leads can be provided, including only one, as long as the number of electrodes is greater than two (including the IPG case function as a case electrode) to allow for lateral steering of the current. Alternatively, a surgical paddle lead can be used in place of one or more of the percutaneous leads. The IPG 102 includes pulse generation circuitry that delivers electrical stimulation energy in the form of a pulsed electrical waveform (i.e., a temporal series of electrical pulses) to the electrodes in accordance with a set of stimulation parameters.

The ETM 105 may also be physically connected via the percutaneous lead extensions 107 and external cable 108 to the neurostimulation lead(s) 101. The ETM 105 may have similar pulse generation circuitry as the IPG 102 to deliver electrical stimulation energy to the electrodes in accordance with a set of stimulation parameters. A programming process may be used to test different parameter sets. The ETM 105 is a non-implantable device that may be used on a trial basis after the neurostimulation leads 101 have been implanted and prior to implantation of the IPG 102, to test the responsiveness of the stimulation that is to be provided. Functions described herein with respect to the IPG 102 can likewise be performed with respect to the ETM 105.

The RC 103 may be used to telemetrically control the ETM 105 via a bi-directional RF communications link 109. The RC 103 may be used to telemetrically control the IPG 102 via a bi-directional RF communications link 110. Such control allows the IPG 102 to be turned on or off and to be programmed with different stimulation parameter sets. The IPG 102 may also be operated to modify the programmed stimulation parameters to actively control the characteristics of the electrical stimulation energy output by the IPG 102. A clinician may use the CP 104 to program stimulation parameters into the IPG 102 and ETM 105 in the operating room and in follow-up sessions.

The CP 104 may indirectly communicate with the IPG 102 or ETM 105, through the RC 103, via an IR communications link 111 or another link. The CP 104 may directly communicate with the IPG 102 or ETM 105 via an RF communications link or other link (not shown). The clinician detailed stimulation parameters provided by the CP 104 may also be used to program the RC 103, so that the stimulation parameters can be subsequently modified by operation of the RC 103 in a stand-alone mode (i.e., without the assistance of the CP 104). Various devices may function as the CP 104. Such devices may include portable devices such as a lap-top personal computer, mini-computer, personal digital assistant (PDA), tablets, phones, or a remote control (RC) with expanded functionality. Thus, the programming methodologies can be performed by executing software instructions contained within the CP 104. Alternatively, such programming methodologies can be performed using firmware or hardware. In any event, the CP 104 may actively control the characteristics of the electrical stimulation generated by the IPG 102 to allow the desired parameters to be determined based on patient feedback or other feedback and for subsequently programming the IPG 102 with the desired stimulation parameters. To allow the user to perform these functions, the CP 104 may include user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g. CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, define the configuration and orientation of the leads, initiate and control the electrical stimulation energy output by the neurostimulation leads, and select and program the IPG with stimulation parameters, including electrode selection, in both a surgical setting and a clinical setting. The external device(s) (e.g., CP and/or RC) may be configured to communicate with other device(s), including local device(s) and/or remote device(s). For example, wired and/or wireless communication may be used to communicate between or among the devices.

An external charger 112 may be a portable device used to transcutaneous charge the IPG 102 via a wireless link such as an inductive link 113. Once the IPG 102 has been programmed, and its power source has been charged by the external charger or otherwise replenished, the IPG 102 may function as programmed without the RC 103 or CP 104 being present.

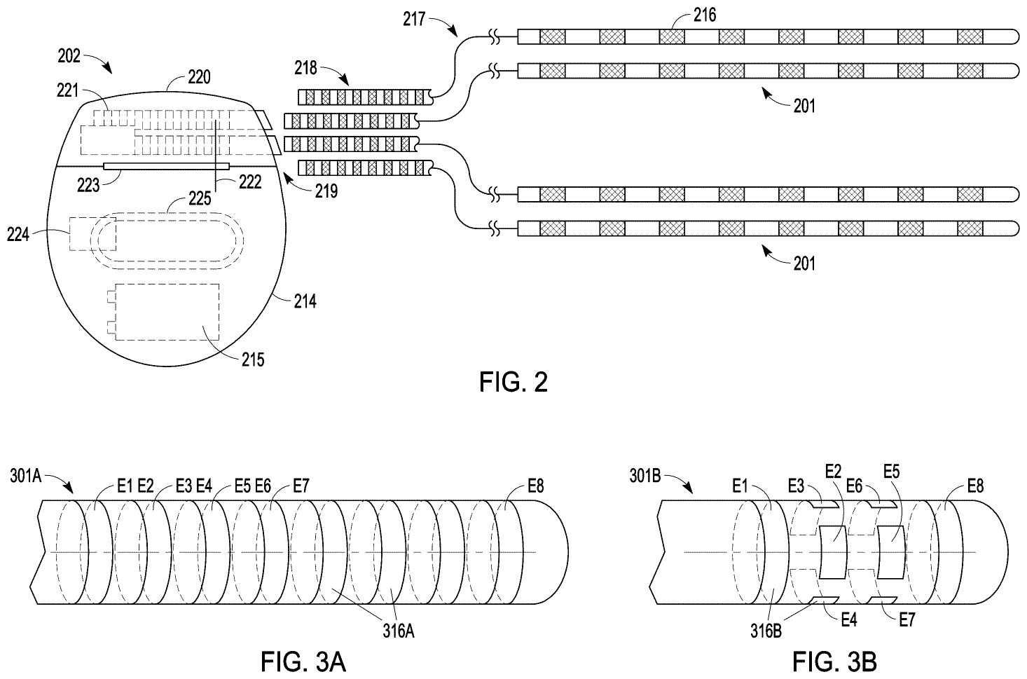

FIG. 2 illustrates, by way of example and not limitation, an IPG 202 in a DBS system. The IPG 202, which is an example of the IPG 102 of the electrical stimulation system 100 as illustrated in FIG. 1, may include a biocompatible device case 214 that holds the circuitry and a battery 215 for providing power for the IPG 202 to function, although the IPG 202 may also lack a battery and may be wirelessly powered by an external source. The IPG 202 may be coupled to one or more leads, such as leads 201 as illustrated herein. The leads 201 may each include a plurality of electrodes 216 for delivering electrostimulation energy, recording electrical signals, or both. In some examples, the leads 201 may be rotatable so that the electrodes 216 may be aligned with the target neurons after the neurons have been located such as based on the recorded signals. The electrodes 216 may include one or more ring electrodes, and/or one or more sets of segmented electrodes (or any other combination of electrodes), examples of which are discussed below with reference to FIGS. 3A and 3B.

The leads 201 may be implanted near or within the desired portion of the body to be stimulated. In an example of operations for DBS, access to the desired position in the brain may be accomplished by drilling a hole in the patient's skull or cranium with a cranial drill (commonly referred to as a burr), and coagulating and incising the dura mater, or brain covering. A lead may then be inserted into the cranium and brain tissue with the assistance of a stylet (not shown). The lead may be guided to the target location within the brain using, for example, a stereotactic frame and a microdrive motor system. In some examples, the microdrive motor system may be fully or partially automatic. The microdrive motor system may be configured to perform actions such as inserting, advancing, rotating, or retracing the lead.

Lead wires 217 within the leads may be coupled to the electrodes 216 and to proximal contacts 218 insertable into lead connectors 219 fixed in a header 220 on the IPG 202, which header may comprise an epoxy for example. Alternatively, the proximal contacts 218 may connect to lead extensions (not shown) which are in turn inserted into the lead connectors 219. Once inserted, the proximal contacts 218 connect to header contacts 221 within the lead connectors 219, which are in turn coupled by feedthrough pins 222 through a case feedthrough 223 to stimulation circuitry 224 within the case 214. The type and number of leads, and the number of electrodes, in an IPG is application specific and therefore can vary.

The IPG 202 may include an antenna 225 allowing it to communicate bi-directionally with a number of external devices. The antenna 225 may be a conductive coil within the case 214, although the coil of the antenna 225 may also appear in the header 220. When the antenna 225 is configured as a coil, communication with external devices may occur using near-field magnetic induction. The IPG 202 may also include a Radio-Frequency (RF) antenna. The RF antenna may comprise a patch, slot, or wire, and may operate as a monopole or dipole, and preferably communicates using far-field electromagnetic waves, and may operate in accordance with any number of known RF communication standards, such as Bluetooth, Zigbee, WiFi, MICS, and the like.

In a DBS application, as is useful in the treatment of tremor in Parkinson's disease for example, the IPG 202 is typically implanted under the patient's clavicle (collarbone). The leads 201 (which may be extended by lead extensions, not shown) may be tunneled through and under the neck and the scalp, with the electrodes 216 implanted through holes drilled in the skull and positioned for example in the subthalamic nucleus (STN) and the pedunculopontine nucleus (PPN) in each brain hemisphere. The IPG 202 may also be implanted underneath the scalp closer to the location of the electrodes' implantation. The leads 201, or the extensions, may be integrated with and permanently connected to the IPG 202 in other solutions.

Stimulation in IPG 202 is typically provided by pulses each of which may include one phase or multiple phases. For example, a monopolar stimulation current may be delivered between a lead-based electrode (e.g., one of the electrodes 216) and a case electrode. A bipolar stimulation current may be delivered between two lead-based electrodes (e.g., two of the electrodes 216). Stimulation parameters typically include current amplitude (or voltage amplitude), frequency, pulse width of the pulses or of its individual phases; electrodes selected to provide the stimulation; polarity of such selected electrodes, i.e., whether they act as anodes that source current to the tissue, or cathodes that sink current from the tissue. Each of the electrodes may either be used (an active electrode) or unused (OFF). When the electrode is used, the electrode may be used as an anode or cathode and carry anodic or cathodic current. The anodic energy contributions may be distributed across more than one anode and the cathodic energy contributions may be distributed across more than one cathode (e.g., electrode fractionalization). Thus, by way of example and not limitation, one electrode may be programmed to provide all (100%) of the anodic energy, and four electrodes may be programmed to provide fractions (e.g., 25%, 25%, 25%, 25%; or 10%, 20%, 30% and 40%) of the total cathodic energy. In some instances, an electrode might be an anode for a period of time and a cathode for a period of time. These and possibly other stimulation parameters taken together comprise a stimulation program that the stimulation circuitry 224 in the IPG 202 may execute to provide therapeutic stimulation to a patient.

In some examples, a measurement device coupled to the muscles or other tissue stimulated by the target neurons, or a unit responsive to the patient or clinician, may be coupled to the IPG 202 or microdrive motor system. The measurement device, user, or clinician may indicate a response by the target muscles or other tissue to the stimulation or recording electrode(s) to further identify the target neurons and facilitate positioning of the stimulation electrode(s). For example, if the target neurons are directed to a muscle experiencing tremors, a measurement device may be used to observe the muscle and indicate changes in, for example, tremor frequency or amplitude in response to stimulation of neurons. Alternatively, the patient or clinician may observe the muscle and provide feedback.

FIGS. 3A-3B illustrate, by way of example and not limitation, leads that may be coupled to the IPG to deliver electrostimulation such as DBS. FIG. 3A shows a lead 301A with electrodes 316A disposed at least partially about a circumference of the lead 301A. The electrodes 316A may be located along a distal end portion of the lead. As illustrated herein, the electrodes 316A are ring electrodes that span 360 degrees about a circumference of the lead 301. A ring electrode allows current to project equally in every direction from the position of the electrode, and typically does not enable stimulus current to be directed from only a particular angular position or a limited angular range around of the lead. A lead which includes only ring electrodes may be referred to as a non-directional lead.

FIG. 3B shows a lead 301B with electrodes 316B including ring electrodes such as E1 at a proximal end and E8 at the distal end. Additionally, the lead 301 also include a plurality of segmented electrodes (also known as split-ring electrodes). For example, a set of segmented electrodes E2, E3, and E4 are around the circumference at a longitudinal position, each spanning less than 360 degrees around the lead axis. In an example, each of electrodes E2, E3, and E4 spans 90 degrees, with each being separated from the others by gaps of 30 degrees. Another set of segmented electrodes E5, E6, and E7 are located around the circumference at another longitudinal position different from the segmented electrodes E2, E3 and E4. Segmented electrodes such as E2-E7 may direct stimulus current to a selected angular range around the lead.

Segmented electrodes may typically provide superior current steering than ring electrodes because target structures in DBS or other stimulation are not typically symmetric about the axis of the distal electrode array. Instead, a target may be located on one side of a plane running through the axis of the lead. Through the use of a radially segmented electrode array, current steering may be performed not only along a length of the lead but also around a circumference of the lead. This provides precise three-dimensional targeting and delivery of the current stimulus to neural target tissue, while potentially avoiding stimulation of other tissue. In some examples, segmented electrodes may be together with ring electrodes. A lead which includes at least one or more segmented electrodes may be referred to as a directional lead. In an example, all electrodes on a directional lead may be segmented electrodes. In another example, there may be different numbers of segmented electrodes at different longitudinal positions.

Segmented electrodes may be grouped into sets of segmented electrodes, where each set is disposed around a circumference at a particular longitudinal location of the directional lead. The directional lead may have any number of segmented electrodes in a given set of segmented electrodes. By way of example and not limitation, a given set may include any number between two to sixteen segmented electrodes. In an example, all sets of segmented electrodes may contain the same number of segmented electrodes. In another example, one set of the segmented electrodes may include a different number of electrodes than at least one other set of segmented electrodes.

The segmented electrodes may vary in size and shape. In some examples, the segmented electrodes are all of the same size, shape, diameter, width or area or any combination thereof. In some examples, the segmented electrodes of each circumferential set (or even all segmented electrodes disposed on the lead) may be identical in size and shape. The sets of segmented electrodes may be positioned in irregular or regular intervals along a length the lead.

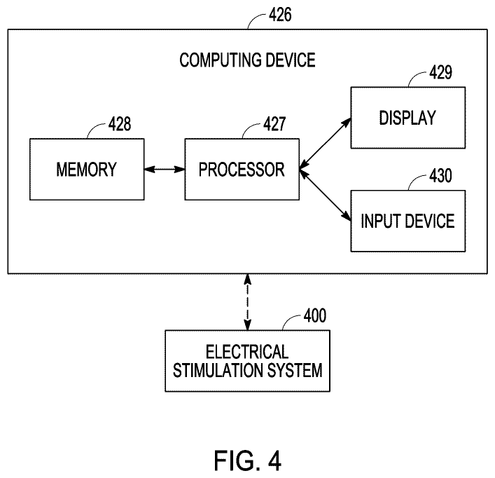

FIG. 4 illustrates, by way of example and not limitation, a computing device 426 for programming or controlling the operation of an electrical stimulation system 400. The computing device 426 may include a processor 427, a memory 428, a display 429, and an input device 430. Optionally, the computing device 426 may be separate from and communicatively coupled to the electrical stimulation system 400, such as system 100 in FIG. 1 Alternatively, the computing device 426 may be integrated with the electrical stimulation system 100, such as part of the IPG 102, RC 103, CP 104, or ETM 105 illustrated in FIG. 1.

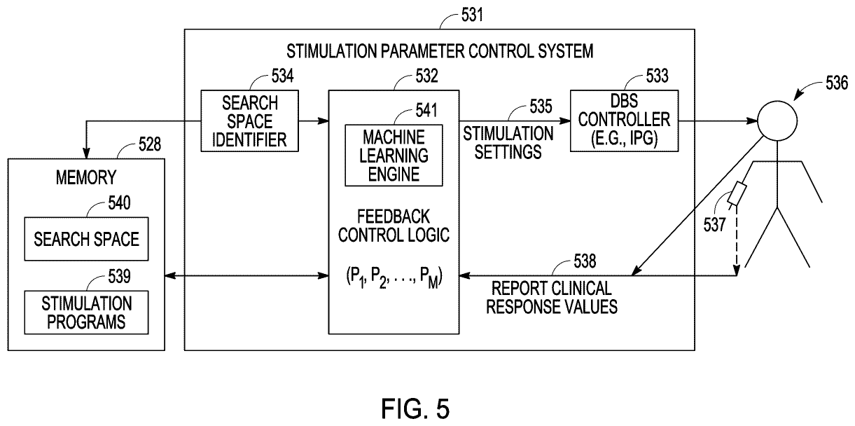

The computing device 426, also referred to as a programming device, may be a computer, tablet, mobile device, or any other suitable device for processing information. The computing device 426 may be local to the user or may include components that are non-local to the computer including one or both of the processor 427 or memory 428 (or portions thereof). For example, the user may operate a terminal that is connected to a non-local processor or memory. In some examples, the computing device 406 may include a watch, wristband, smartphone, or the like. Such computing devices may wirelessly communicate with the other components of the electrical stimulation system, such as the CP 104, RC 103, ETM 105, or IPG 102 illustrated in FIG. 1. The computing device 426 may be used for gathering patient information, such as general activity level or present queries or tests to the patient to identify or score pain, depression, stimulation effects or side effects, cognitive ability, or the like. In some examples, the computing device 426 may prompt the patient to take a periodic test (for example, every day) for cognitive ability to monitor, for example, Alzheimer's disease. In some examples, the computing device 426 may detect, or otherwise receive as input, patient clinical responses to electrostimulation such as DBS, and determine or update stimulation parameters using a closed-loop algorithm based on the patient clinical responses, as described below with reference to FIG. 5. Examples of the patient clinical responses may include physiological signals (e.g., heart rate) or motor parameters (e.g., tremor, rigidity, bradykinesia). The computing device 426 may communicate with the axis. CP 104, RC 103, ETM 105, or IPG 102 and direct the changes to the stimulation parameters to one or more of those devices. In some examples, the computing device 426 may be a wearable device used by the patient only during programming sessions. Alternatively, the computing device 426 may be worn all the time and continually or periodically adjust the stimulation parameters. In an example, the closed-loop algorithm for determining or updating stimulation parameters may be implemented in a mobile device, such as a smartphone, that is connected to the IPG or an evaluating device (e.g., a wristband or watch). These devices may also record and send information to the clinician.

The processor 427 may include one or more processors that may be local to the user or non-local to the user or other components of the computing device 426. In an example, the processor 427 may execute instructions (e.g., stored in the memory 428) to determine a search space of electrode configurations and parameter values, and identify or update one or more stimulation settings that are selectable for use in electrostimulation therapies such as DBS. The search space may include a collection of available electrodes, possible electrode configurations, and possible values or value ranges of one or more stimulation parameters that may be applied to selected electrodes to deliver electrostimulation. The search space may be specific to a particular lead or a type of lead with respect to a specific neural target. As a result, for different leads or types of lead and/or for different neural targets, the processor 427 may determine respective different search spaces. A stimulation setting includes an electrode configuration and values for one or more stimulation parameters. The electrode configuration may include information about electrodes (ring electrodes and/or segmented electrodes) selected to be active for delivering stimulation (ON) or inactive (OFF), polarity of the selected electrodes, electrode locations (e.g., longitudinal positions of ring electrodes along the length of a non-directional lead, or longitudinal positions and angular positions of segmented electrodes on a circumference at a longitudinal position of a directional lead), stimulation modes such as monopolar pacing or bipolar pacing, etc. The stimulation parameters may include, for example, current amplitude values, current fractionalization across electrodes, stimulation frequency, stimulation pulse width, and like.

The processor 427 may identify or modify a stimulation setting from the search space through an optimization process until a search criterion is satisfied, such as until an optimal, desired, or acceptable patient clinical response is achieved. Electrostimulation programmed with a setting may be delivered to the patient, clinical effects (including therapeutic effects and/or side effects, or motor symptoms such as bradykinesia, tremor, or rigidity) may be detected, and a clinical response may be evaluated based on the detected clinical effects. When actual electrostimulation is administered, the settings may be referred to as tested settings, and the clinical responses may be referred to as tested clinical responses. In contrast, for a setting in which no electrostimulation is delivered to the patient, clinical effects may be predicted using a computational model based at least on the clinical effects detected from the tested settings, and a clinical response may be estimated using the predicted clinical effects. When no electrostimulation is delivered the settings may be referred to as predicted or estimated settings, and the clinical responses may be referred to as predicted or estimated clinical responses.

In various examples, portions of the functions of the processor 427 may be implemented as a part of a microprocessor circuit. The microprocessor circuit may be a dedicated processor such as a digital signal processor, application specific integrated circuit (ASIC), microprocessor, or other type of processor for processing information. Alternatively, the microprocessor circuit may be a processor that may receive and execute a set of instructions of performing the functions, methods, or techniques described herein.

The memory 428 may store instructions executable by the processor 427 to perform various functions including, for example, determining a reduced or restricted electrode configuration and parameter search space (also referred to as a “restricted search space”), creating or modifying one or more stimulation settings within the restricted search space, etc. The memory 428 may store the search space, the stimulation settings including the “tested” stimulation settings and the “predicted” or “estimated” stimulation settings, clinical effects (e.g., therapeutic effects and/or side effects) and clinical responses for the settings, and/or instructions for implementing a testing process for testing stimulation parameters. The memory 428 may be a computer-readable storage media that includes, for example, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which may be used to store the desired information, and which may be accessed by a computing device.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, Bluetooth™, near field communication, and other wireless media.

The display 429 may be any suitable display or presentation device, such as a monitor, screen, display, or the like, and may include a printer. The display 429 may be a part of a user interface configured to display information about stimulation settings (e.g., electrode configurations and stimulation parameter values and value ranges) and user control elements for programming a stimulation setting into an IPG.

The input device 430 may be, for example, a keyboard, mouse, touch screen, track ball, joystick, voice recognition system, or any combination thereof, or the like. Another input device 430 may be a camera from which the clinician may observe the patient. Yet another input device 430 may a microphone where the patient or clinician may provide responses or queries.

The electrical stimulation system 400 may include, for example, any of the components illustrated in FIG. 1. The electrical stimulation system 400 may communicate with the computing device 426 through a wired or wireless connection or, alternatively or additionally, a user may provide information between the electrical stimulation system 400 and the computing device 426 using a computer-readable medium or by some other mechanism.

FIG. 5 illustrates, by way of example and not limitation, a stimulation parameter control system and a part of the environment in which it may operate. The stimulation parameter control system 531, which may be implemented as a part of the processor 427 in FIG. 4, may include a feedback control logic 532, a DBS controller 533, and a search space identifier 534. DBS is used as an example. It is noted that the system may be implemented for other stimulation therapies such as, but not limited to, SCS or PNS. The feedback control logic 532 may be implemented in, for example, the CP 104 or the RC 103 in FIG. 1. The feedback control logic 532 may determine or modify one or more stimulation settings 535 for a stimulation lead at a target stimulation region, such as a region in a brain hemisphere. A stimulation setting may include an electrode configuration and values for one or more stimulation parameters (P₁, P₂, . . . , Pₘ) 535. The electrode configuration includes information about electrodes (ring electrodes and/or segmented electrodes) selected to be active for delivering stimulation (ON) or inactive (OFF), polarity of the selected electrodes, electrode locations (also referred to as contact locations, which may include longitudinal positions of ring electrodes along the length of a lead, or angular positions of segmented electrodes about a circumference of a cross-section of the lead at a longitudinal position), and stimulation modes (e.g., monopolar pacing or bipolar pacing), etc. The stimulation parameters may include, for example, current amplitude values, current fractionalization across electrodes, stimulation frequency, stimulation pulse width, etc. In some examples, the feedback control logic 532 may modify the stimulation setting 535 such as by changing a stimulation parameter value, or modifying an electrode configuration.

The stimulation setting 535 may be provided to the DBS controller 533 to configure the IPG or ETM to deliver DBS therapy to the patient 536 in accordance with the stimulation setting or the modified stimulation setting. The stimulation may produce certain therapeutic effects and/or side effects on the patient 536. Such therapeutic effectiveness and side effects, also referred to as clinical responses or clinical metrics, may be provided to the feedback control logic 532. In an example, the clinical responses may be based on patient or clinician observations. For example, motor symptoms such as bradykinesia (slowness of movement), rigidity, tremor, among other symptoms or side effects, may be scored by the patient or by the clinician upon overserving or questioning the patient. In some examples, the clinical responses may be objective in nature, such as measurements automatically or semi-automatically taken by a sensor 537. In an example, the sensor 537 may be included in a wearable device associated with patient 536, such as a smart watch. For example, a Parkinson's patient may be fitted with a wearable sensor that measures tremors, such as by measuring the frequency and amplitude of such tremors.

The clinical responses, either reported by the patient or measured by a sensor, may be converted to clinical response values 538, also referred to as clinical response scores. In an example, the clinical response values 538 may be computed based on the intensity, frequency, or duration of one or more of tremor, rigidity, or bradykinesia responses. Based upon the received clinical response values 538, the feedback control logic 532 may adjust electrode configurations or values of one or more stimulation parameters 535. The feedback control logic 532 may send the adjusted (new or revised) stimulation setting 535, such as the electrode configuration or the adjusted stimulation parameter values, to further configure the DBS controller 533 to change the stimulation parameters of the leads implanted in patient 506 to the adjusted values.

The feedback-control loop as illustrated in FIG. 5 may continue until an optimal, desired, or acceptable outcome is reached, such as maximizing therapeutic effectiveness while minimizing unwanted side effects, or until a specific stop condition is reached such as number of iterations, time spent in programming session, or the like. An outcome may be considered optimal, desired, or acceptable if it meets certain threshold values or tests (e.g., improved clinical response for the patient, faster programming of the device, increased battery life, and/or control multiple independent current sources and directional lead). Such an iterative process of looking for a stimulation setting (e.g., an electrode configuration and stimulation parameter values for the electrode) is referred to as a stimulation setting optimization process. The outcome being reached may be referred to as an optimization criterion, and the resultant stimulation setting may be referred to as an optimal base stimulation setting (BSS). By way of example and not limitation, the optimization criterion may include possible optimal clinical outcome within the parameters chosen; time spent, iterations taken, or power usage to explore the search space until a desired clinical outcome is reached (assuming multiple outcomes with the same or comparable clinical response); among others.

In an example, the optimization criterion includes the clinical response values 538 exceeding a threshold value or falling into a specified value range, indicating a satisfactory therapeutic outcome has reached. Depending on how the clinical response values are computed, one or more optimal base stimulation settings may be determined. For example, the clinical response values may be computed using a single response effect (e.g., one of bradykinesia, tremor, or rigidity). Accordingly, three optimal base stimulation settings may be generated: a first optimal base stimulation setting (BSS₁) corresponding to a bradykinesia score exceeding a threshold, a second optimal base stimulation setting (BSS₂) corresponding to a tremor score exceeding a threshold, and a third optimal base stimulation setting (BSS₃) corresponding to a rigidity score exceeding a threshold. In another example, the clinical response values may be a composite score computed as a weighted combination of multiple clinical effects, such as a %*bradykinesia+b %*tremor+c %*rigidity. Accordingly, a fourth optimal base stimulation setting (BSS₄) may be generated, corresponding to the composite clinical response score exceeding a threshold. In some examples, the stimulation setting optimization may be performed in an in-clinic programming session such during implantation or revision of a DBS system or device follow-up.

The optimal base stimulation settings (e.g., BSS₁ through BSS₄), may be stored in the memory 528. In an example, a stimulation setting, along with the corresponding unique clinical response indicator (e.g., weighted combination of clinical effects with unique weight factors) form a stimulation program 539, which may also be stored in the memory 404. Each stimulation programmed may be associated with, or tagged by, one or more unique clinical response indicators. In some examples, the clinical response values 538 may be weighted according to the time at which the test took place.

In various examples, the stimulation parameter control system 531 may be executed on its own and is not connected to a controller. In such instances it may be used to merely determine and suggest programming parameters, visualize a parameter space, test potential parameters, etc.

The process of searching for a stimulation setting (e.g., an electrode configuration and/or stimulation parameter values) typically involves significant computation and time, especially when electrode configuration involves segmented electrodes in a directional lead. If testing all possible settings in the entire parameter space (including electrode configurations and combinations of stimulation parameter values) is done as comprehensively as possible, stimulation would need to be provided to the patient for each possible setting, which may end up with a burdensome and time-consuming programming session. Because practically a programming session may only last a few hours, only a fraction of possible electrode configuration and stimulation parameter combinations may reasonably be tested and evaluated. To reduce the time taken and to improve the efficiency of stimulation setting optimization process, a reduced or restricted electrode configuration and parameter search space may be used. By applying limitations or constraints to the electrode configurations and parameter values, the restricted search space may include a subset of electrodes (e.g., a subset of ring electrodes and/or a subset of segmented electrodes on a lead) that are selected as active electrodes for delivering stimulation, and values or value ranges for one or more stimulation parameters (e.g., a range of current amplitude ranges for an active electrode). Stimulation setting optimization, when performed within such a search space, may be more efficient and cost-effective than searching through the entire parameter space for one or more optimal base stimulation settings such as BSS₁-BSS₄ as discussed above. Identifying a search space may involve identifying a variable ramp for testing increasing adjustable parameter values.

The search space identifier 534 may automatically determine a search space 540 for a stimulation lead at a neural target, such as a region in a brain hemisphere, by imposing certain limitations or constraints on the electrode configurations and/or parameter values or value ranges. In an example, the search space 540 may be determined based on spatial information of the lead, such as lead positions with respect to neural targets, which may be obtained from imaging data of the lead and patient anatomy. Additionally, or alternatively, the search space 540 may be determined based on physiological information such as physiological signals sensed by the electrodes at their respective tissue contact locations. The physiological information may include patient clinical responses to stimulation. In some examples, prior knowledge about patient medical condition, health status, DBS treatment history may be used to determine the search space 540. In an example, the search space identifier 534 may exclude those electrodes on the lead that are out of a region of interest, such that the search space includes only those electrodes within the target of interest. One or more stimulation parameters may be restricted to take certain values or within value ranges. For example, the restricted search space may include certain electrode positions and value ranges for stimulation current amplitude, frequency, or pulse width. The feedback control logic 532 may determine one or more optimal base stimulation settings (e.g., BSS₁-BSS₄) by searching through the identified search space 540. The identified search 540 may be stored in the memory 528.

The feedback control logic 532 may include a machine learning engine 541 that may facilitate the stimulation parameter control system 531 (or a user of the system) to explore the search space in order to choose values for programming the DBS controller 533. The machine learning engine 541 may employ supervised or unsupervised learning algorithms to train a prediction model, and use the trained prediction model to predict patient clinical responses to an untested stimulation setting (e.g., untested stimulation parameter values or untested electrode configurations), or to estimate or predict stimulation parameters values or electrode configurations that, when provided to the DBS controller 533 to deliver stimulation accordingly to the patient 536, would produce desired or improved clinical responses. Examples of the learning algorithms include, for example, Naive Bayes classifiers, support vector machines (SVMs), ensemble classifiers, neural networks, Kalman filters, regression analyzers, etc. The machine learning engine 541 may build and train a prediction model using training data, such as stimulation parameter values and corresponding patient clinical responses. The training data may be acquired from a training session such as performed in a clinic. Additionally, or alternatively, the training data may be obtained from historical data acquired by the stimulation parameter control system 531. With its learning and prediction capability, the machine learning engine 541 may aid a user (e.g., a clinician) in exploring the stimulation parameter space more effectively and more efficiently to produce results that are optimal, desired, or acceptable.

In some examples, the machine learning engine 541 may use imaging data to inform the choice of the next set of values, which may be used when the algorithm finds itself in a region of parameter space for which the clinical responses are not substantially affected by the changes in the stimulation parameters, and the choice of next step is not apparent from the patient response alone. Imaging data that provides information about the location of the lead in the patient's brain along with priors informing the algorithm of which directions may be better choices for the next step could lead to faster convergence.

In some examples, the machine learning engine 541 may determine expected outcomes for parameter values that have not yet been tested based upon what the machine learning engine 541 has “learned” thus far, and provide a recommendation for a next set of values to test. Here, testing refers to the iterative testing required to find an optimal stimulation setting for configuring the DBS controller 533. The recommendation for a next set of values to test is based upon which of the determined expected outcomes meet a set of designated (determined, selected, preselected, etc.) criteria (e.g., rules, heuristics, factors, and the like). For example, rules considered may include such factors as: the next set of values may not be one of the last 10 settings tested or may not be too close to previously tested setting. Accordingly, the feedback control logic 532 with its machine learning engine 541 is used to systematically explore the stimulation parameter space based upon what it has learned thus far and (optionally) different rules and/or heuristics that contribute to achieving optimal outcomes more efficiently.

The process for determining expected outcomes for parameter values that have not yet been tested may involve use of other data for machine learning. For example, data from other programming sessions for the same patient as well as from other patients may be used to train the machine learning engine 541. In some examples, no prior data may be used. In this case, the machine learning engine 541 may use data learned from this patient only in one particular setting. In other examples, data from the same patient but from previous sessions may be used. In some examples all patient data from all sessions may be used. In some examples all patient data utilizing lead location information (knowledge of lead location in space relative to anatomy) may be used. Different other combinations are also possible.

In order to use this data for machine learning purposes, the data may first be cleansed, optionally transformed, and then modeled. In some examples, new variables are derived, such as for use with directional leads, including central point of stimulation, maximum radius, spread of stimulation field, or the like. Data cleansing and transformation techniques such as missing data imputation and dimension reduction may be employed to prepare the data for modeling.

The machine learning engine 541 may determine how best a predicted outcome meets the optimal outcome metrics. Various optimization techniques may be used, examples of which may include but are not limited to: optimization algorithms and estimation procedures used to fit the model to the data (e.g., gradient descent, Kalman filter, Markov chain, Monte Carlo, and the like); optimization algorithms reformulated for search (e.g., simulated annealing); spatial interpolation (e.g., kriging, inverse distance weighting, natural neighbor, etc.); supplementary methods that aid the optimization process (e.g., variable selections, regularization, cross validation, etc.); other search algorithms (e.g., golden-section search, binary search, etc.). Using any of these techniques, the machine learning engine 541 may decide whether a particular predicted outcome for a set of stimulation parameter values is the fastest sufficing outcome, the best possible clinical outcome, or the optimal outcome with least battery usage, for example.

The feedback control logic 532 may be used to search and configure different types of stimulation parameters of the various leads potentially causing different clinical effects upon the patient 536. Examples of the stimulation parameters may include electrode configurations (electrode selection, polarities, monopolar or bipolar modes of stimulation), current fractionalization, current amplitude, pulse width, frequency, among others. Given these possible stimulation parameters, the stimulation parameter control system 531 may move about the parameter space in different orders, by different increments, and limited to specific ranges. In some examples, the stimulation parameter control system 531 may allow the user to provide search range limitations to one or more of the stimulation parameters to limit the range for that stimulation parameter over which the system will search for parameters. For example, the user may restrict which electrodes may be used for stimulation or may restrict the amplitude or pulse width to a certain range or with a selected maximum or minimum. As one illustration, based on the site of implantation, the user may be aware that the distal-most and proximal-most electrodes are unlikely to produce suitable stimulation and the user limits the range of electrodes to exclude these two electrodes.

For a lead with segmented electrodes, the number of possibilities for parameter selection may be very large when combinations of electrodes and different amplitudes on each electrode are possible. In some examples using a lead with segmented electrodes, the selection of electrodes used for stimulation may be limited to fully directional selections (i.e., selection of only a single segmented electrode) and fully concentric selections (i.e., all electrodes in a single set of segmented electrodes are active with the same amplitude). In other examples, the initial movement through parameter space may be limited to fully directional and fully concentric selections. After a set of stimulation parameters is identified using these limits, variation in the selection of electrodes may be opened up to other possibilities near the selection in the identified set of stimulation parameters to further optimize the stimulation parameters.

In some examples, the number of stimulation parameters that are varied and the range of those variations may be limited. For example, some stimulation parameters (e.g., electrode selection, amplitude, and pulse width) may have larger effects when varied than other stimulation parameters (e.g., pulse shape or pulse duration). The movement through stimulation parameter space may be limited to those stimulation parameters which exhibit larger effects. In some examples, as the stimulation parameter control system 531 proceeds through testing of sets of stimulation parameters, the system may observe which stimulation parameters provide larger effects when varied and focus on exploring variation in those stimulation parameters.

In some examples, the stimulation parameter control system 531 may include a user interface for visualizing exploration of the stimulation parameter space as the system determines new and better parameter values to test until a solution is determined that fits within certain designated thresholds or a stop condition is reached. In some examples of the stimulation parameter control system 531, the user interface is part of the feedback control logic. In other examples, the user interface may be part of another computing system that is part of the stimulation parameter control system 531 or may be remote and communicatively connected to the stimulation parameter control system 531. The user interface may present to a user (such as a clinician, physician, programmer, etc.) a visualization of the predicted expected outcomes for (some of) the stimulation parameter values not yet tested and a recommendation for the next set of stimulation parameter values to test.

In some examples where a deep brain stimulator is configured via the DBS controller 533 with at least one set of stimulation parameter values forwarded by the feedback control logic 532, the clinician may monitor the patient throughout the process and record clinical observables in addition to the patient 536 being able to report side effects. When a side effect is observed, the various search algorithms may take that fact into account when selecting/suggesting a next set of values to test. In some examples, for example, those that select contacts via monopolar review, other parameters may be changed until they cause a side effect, which case is noted as a boundary. For example, in monopolar review where amplitude is another stimulation parameter being varied, the amplitude may be increased progressively (e.g., via a variable ramp protocol) until a side-effect is observed.

In some examples, more than one clinical metric (e.g., tremor, rigidity, bradykinesia, etc.) may be important observables. Different examples of the stimulation parameter control system 531 may handle these metrics differently. For example, some examples might identify an ideal location for each metric and choose one ideal location between them, set in the patient's remote controller so the patient may choose as needed, or chose a best combined outcome. As another example, some examples may search multiple outcomes at the same time and use the best combined score as the best outcome or find a best location for each metric individually. As yet another example, some examples may use a sequential process for selecting stimulation parameter values for multiple outcomes. For example, a system may search parameter space for a first outcome (e.g., bradykinesia) and, upon finding a suitable end condition, then search parameter space for a second outcome (e.g., rigidity). While searching parameter space for the first outcome, clinical response values for both the first and second outcomes may be obtained. Thus, when the system switches to the second outcome there are already a number of clinical response values for that outcome which will likely reduce the length of the search.

In some examples, two stimulation leads may be implanted to produce stimulation effects on two sides of the body (e.g., the right and left sides of the body). The same procedure described herein may be used to either jointly determine the stimulation parameters for the two leads by exploring the joint parameter space or individually determine stimulation parameters for the two leads by exploring the parameter space for each lead individually. In some examples, the user may determine for each side of the body which clinical response is dominant or most responsive. This may be done, for example, by having the patient perform a single task which captures multiple responses (e.g., connecting dots on the screen to monitor tremor and bradykinesia of the movement) or a small series of tasks. This enables the system to determine which clinical response to use to identify the stimulation parameters for that side of the body.

As noted, the feedback may be provided directly by the patient 536, entered by an observer such as a clinician (not shown), or may be provided by means of a sensor 537 associated with and in physical, auditory, or visual contact with the patient 536. Examples may include, but are not limited to, accelerometers, microphones, and cameras. In an example, the sensor 537 may be included in a wearable device associated with patient 536, such as a smart watch. In an example where the feedback may be monitored automatically or semi-automatically, such as with use of sensor 537, it may not be necessary for a clinician or other observer to be present to operate the stimulation parameter control system 531. Accordingly, in such examples a user interface may not be present in system 531.

In some examples, the stimulation parameter control system 531 may determine one or more optimal base stimulation settings using predicted clinical responses for untested stimulation parameter values or untested electrode configurations without actually delivering stimulation. Such base stimulation settings are referred to as estimated or predicted base stimulation settings, to distinguish from the tested base stimulation settings (e.g., BSS₁-BSS₄) that are based on the tested clinical response (either reported by the patient or measured by a sensor) to actually delivered stimulation. For examples, based on the “tested” base stimulation settings BSS₁-BSS₄, the stimulation parameter control system 531 may estimate an optimal base stimulation setting associated with a composite clinical response defined as x %*bradykinesia+y %*tremor+z %*rigidity, or simply denoted by the weight factors (x %, y %, z %). By way of example and not limitation, the stimulation parameter control system 531 may generate a fifth optimal base stimulation setting (BSS₅) corresponding to a composite clinical response using bradykinesia and tremor only, each weighted 50%; a sixth optimal base stimulation setting (BSS₆) corresponding to a composite clinical response using tremor and rigidity only, each weighted 50%; a seventh optimal base stimulation setting (BSS₇) corresponding to a composite clinical response using bradykinesia and rigidity only, each weighted 50%; or an eighth optimal base stimulation setting (BSS₈) corresponding to a composite clinical response using bradykinesia, tremor, and rigidity weighted 40%, 40%, and 20%, respectively. Similar to the tested base stimulation settings BSS₁-BSS₄, the estimated base stimulation settings BSS₅-BSS₈, associated with their respective clinical response indicators (e.g., weight factors for clinical effects), may be stored in the memory 528 as respective stimulation programs 539. In an example, the stimulation programs 539 may be stored in a lookup table, where each tested or estimated base stimulation setting (e.g., BSS₁ through BSS₈) may be tagged by respective clinical response indicators or weight factors for clinical effects. In an example, the memory 528 may be a part of memory circuitry internal to the IPG. The RC or the CP may request access to the memory 528 to retrieve therefrom one or more stored stimulation programs 539 or the search space 540.

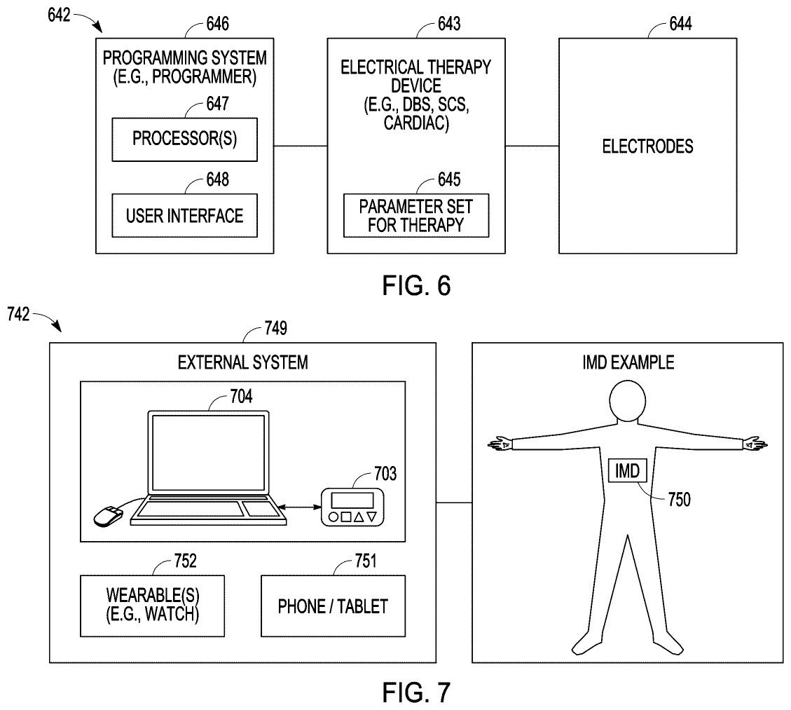

FIG. 6 illustrates, by way of example, an example of an electrical therapy-delivery system. The illustrated system 642 includes an electrical therapy device 643 configured to deliver an electrical therapy to electrodes 644 to treat a condition in accordance with a programmed parameter set 645 for the therapy. The system 642 may include a processing system 646 that may include one or more processors 647 and a user interface 648, which may be used to program and/or evaluate the parameter set(s) used to deliver the therapy. The illustrated system 642 may be a DBS system for treating a movement disorder, such as has been illustrated and discussed with respect to FIGS. 1-5, and/or a system for monitoring the movement disorder.

In some embodiments, the illustrated system 642 may include an SCS system to treat pain and/or a system for monitoring pain. By way of example, a therapeutic goal for conventional SCS programming may be to maximize stimulation (i.e., recruitment) of the dorsal column (DC) fibers that run in the white matter along the longitudinal axis of the spinal cord and minimal stimulation of other fibers that run perpendicular to the longitudinal axis of the spinal cord (e.g., dorsal root fibers).

FIG. 7 illustrates, by way of example and not limitation, the electrical therapy-delivery system of FIG. 6 implemented using an implantable medical device (IMD). The illustrated system 742 includes an external system 749 that may include at least one programming device. The illustrated external system 749 may include a clinician programmer 704, similar to CP 104 in FIG. 1, configured for use by a clinician to communicate with and program the neuromodulator, and a remote control device 703, similar to RC 103 in FIG. 1, configured for use by the patient to communicate with and program the neuromodulator. For example, the remote control device 703 may allow the patient to turn a therapy on and off and/or may allow the patient to adjust patient-programmable parameter(s) of the plurality of stimulation parameters. FIG. 7 illustrates an IMD 750, although the monitor and/or therapy device may be an external device such as a wearable device. The external system 749 may include a network of computers, including computer(s) remotely located from the IMD 750 that are capable of communicating via one or more communication networks with the programmer 704 and/or the remote control device 703. The remotely located computer(s) and the IMD 750 may be configured to communicate with each other via another external device such as the programmer 704 or the remote control device 703. The remote control device 703 and/or the programmer 704 may allow a user (e.g., patient and/or clinician or rep) to answer questions as part of a data collection process. The external system 749 may include personal devices such as a phone or tablet 751, wearables such as a watch 752, sensors or therapy-applying devices. The watch may include sensor(s), such as sensor(s) for detecting activity, motion and/or posture. Other wearable sensor(s) may be configured for use to detect activity, motion and/or posture of the patient. The external system 749 may include, but is not limited to, a phone and/or a tablet. The phone and/or tablet may include camera(s), microphone(s), accelerometer(s) or other sensors that can be used to provide feedback.

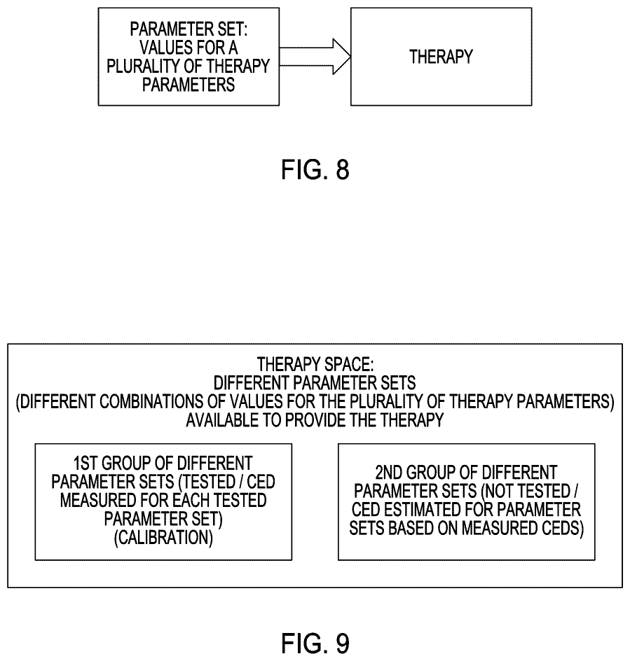

FIG. 8 illustrates a therapy being delivered according to a parameter set. The parameter set may be programmed into the device to deliver the specific therapy using specific values for a plurality of therapy parameters. For example, the therapy parameters that control the therapy may include pulse amplitude, pulse frequency, pulse width, and electrode configuration (e.g., selected electrodes, polarity and fractionalization). The parameter set includes specific values for the therapy parameters.

FIG. 9 illustrates a therapy space, which includes different parameter sets potentially available for delivering the therapy. The different parameter sets have unique combinations of values for the therapy parameters. The therapy space may be burdensomely large as there may be many unique combinations of values for therapy parameters (e.g., many unique parameter sets). Some parameter sets within the therapy space may be tested and the corresponding clinical effect data (CED) may be measured or otherwise acquired for the tested parameter sets. A variable ramping protocol for ramping a parameter may be used to identify the parameter sets that are tested. These tested parameter sets are illustrated as a first group of different parameter sets within the therapy space. Other parameter sets may not be tested (e.g., second group of different parameter sets). The CED for these parameter sets may be estimated based on measured CEDs for the patient or a patient population.

For example, CED may be directly measured to provide calibration settings. The therapy sessions may be delivered using different therapy settings, and the CED may be recorded for each session. For a neurostimulator such as DBS, SCS, PNS or TENS, for example, the therapy may involve delivering electrical waveforms, which may be a pulsed waveform. Programmable settings for the pulse waveform may include a pulse amplitude, a pulse width, a pulse frequency, a pulse train duration, a pulse-to-pulse duty cycle, a pulse train to pulse train duty cycle (stimulation ON/OFF), and a stimulation schedule (e.g., programmable start and/or stop times, such as but not necessarily a calendar-based schedule). The programmable settings may further include controlling which of a plurality of electrodes are active and which are off, the polarity of each active electrode (which active electrode(s) are anode(s) and which are cathode(s), and the contributions (e.g., electrode fractionalization) of total energy delivered to individual one(s) of the anode(s) and individual one(s) of the cathode(s). Thus, by way of example and not limitation, one electrode may be programmed to provide all (100%) of the anodic energy, and four electrodes may be programmed to provide fractions (e.g., 25%, 25%, 25%, 25%; or 10%, 20%, 30% and 40%) of the total cathodic energy. Controlling the individual contributions by individual electrodes adjusts the location and shape of the stimulation field, to modulate different combinations of neural elements. The settings may be spread throughout the stimulation space for use in identifying clinical responses from session to session, including both the previously-measured clinical effects for tested stimulation settings and predicted or estimated clinical effects for stimulation settings that were not previously tested.

Programming neurostimulation parameter settings for a patient attempts to balance the competing pressures of taking sufficient time to deliver a good therapy while avoiding patient discomfort during the programming session and to make the programming process as swift as possible for both the clinicians and patients. Programming may test a stimulation parameter set by simply turning it on. However, it is more comfortable to the patient to increase stimulation more gradually. Programming may involve a clinician manually controlling a rate of increase. The system may be designed to test different parameter sets by keeping values for most of the parameters static and changing values for one or more parameters (e.g., an adjustable parameter within the parameter set) to determine the stimulation effect for the different values. For example, some systems may use static ramping protocols that increase stimulation amplitude uniformly to the new target.

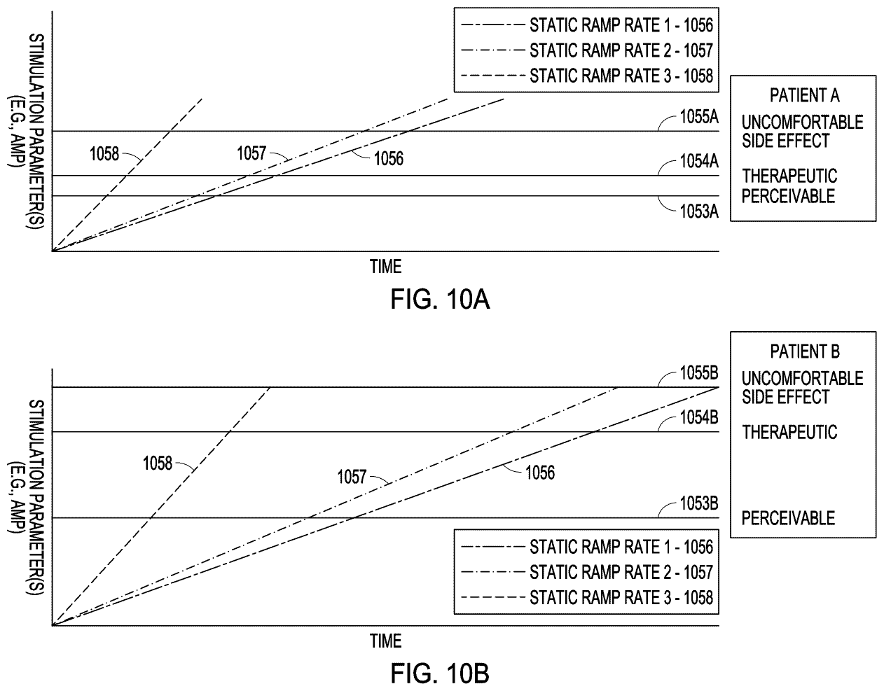

FIGS. 10A-10B illustrate, by way of example and not limitation, the use of static ramping protocols for two patients who experience some stimulation effects differently. For example, the first patient may experience perceivable stimulation 1053A, 1054A, 1055A, therapeutic stimulation and uncomfortable stimulation at three different amplitudes that are relatively close together as illustrated in FIG. 10A, and the second patient may experience perceivable stimulation 1053B, therapeutic stimulation 1054B and uncomfortable stimulation 1055B differently at amplitudes that are relatively far apart as illustrated in FIG. 10B. For the patient illustrated in 10A, faster ramping may cause the patient discomfort if the testing quickly causes side effects after it is determined to be therapeutically effective. This may make it different to try to find a therapy where the side effect threshold is significantly larger than the therapeutic threshold. For the patient illustrated in 10B, the slower rate in particular may take too long for the patient and/or clinician to reach the threshold(s) of interest.

A known process implemented by DBS programming software attempts to provide this balance and reduce repetitive button clicks using an automated ramp tool during the programming process. The automated ramp tool allows a user to test neurostimulation settings by ramping at one of three static speeds to a target stimulation amplitude. For example, a system may include one or more static ramps that may be selected. For example, the static ramping protocols may steadily increase amplitude by 0.1 mA per second 1056 throughout the ramping protocol, may be a little faster by steadily increasing amplitude by 0.2 mA per second 1057 throughout the ramping protocol, or may be even faster by steadily increasing amplitude by 0.5 mA per second 1058 throughout the ramping protocol.

A patient may be expected to experience a side effect at a certain level, but there is patient-to-patient variability in the level where a side effect will occur, there are variations where the side effect occurs in the same patient based on different stimulation locations and responses to different stimulation parameters. Therefore, ramping is used because it is considered to be statistically probable that a side effect may occur near a certain amplitude. For example, in an amplitude scale from 0 to 100, the amplitude level of 70 may be associated with an 80% chance of the patient experiencing a side effect. However, some patients may never have a side effect, and some patients may be extra sensitive to stimulation and may get a side effect at an amplitude level around 40 or 50. There are other reasons for ramping. For example, jumping directly up to the amplitude level of 70 may be uncomfortable for the patient. Because it may take a while for stimulation to “wash-in” or impact the patient, it may not be effective to take a big step up to an amplitude level of 50 and then stepping to 70 Also, some patients have a hard to time communicate so they may not be able to timely communicate that the perception or distribution even if the patient felt it instantaneously. In this example where there is an amplitude scale from 0 to 100 and the amplitude level of 70 is associated with an 80% chance of the patient experiencing a side effect, the side effect that occurs at the amplitude level 70 may not occur immediately. If the amplitude continues to quickly increase, the amplitude may reach 100 before the side effect is perceived. Because the amplitude level is at 100, the side effect may be very uncomfortable and may take a significant period of time for the side effect to go away even if the stimulation is completely turned off.

Comfort is a main determining factor for determining an appropriate ramp speed. For example, a fast ramp may be used if the stimulation is imperceptible and a slow ramp may be used if the stimulation is perceptible and nearing a level of discomfort. Patients may become exhausted if levels of significant discomfort are often reached, which makes it more difficult to program.

This present subject matter proposes a more intelligent variable ramp that allows for faster amplitude ramping under established safer conditions to a set threshold, and slower ramping beyond that threshold in regions where large/rapid stimulation changes can induce significant patient discomfort. The variable ramp technique is useful to balance time and results by balancing the desire of clinicians to test as many patients as quickly as possible along with the patient's desire to quickly proceed with the programming process to obtain a therapeutically effective therapy against the desire to limit side effects, and the severity of the side effects, that may occur during testing when programming the therapy. Also, the technique is useful to help inform clinicians with the programming process, and may be particularly useful for clinicians who have less experience with programming different therapies for many different patients.

The variable ramp for changing amplitude values (or values for other parameter(s)) may be configured to balance the desire to accurately test different values for a parameter set in a shortened period of time without causing patient discomfort. For example, the intelligent ramp may allow for faster amplitude ramping under established safer conditions to a set threshold, and may allow slower ramping beyond that threshold in regions where large/rapid stimulation changes can induce significant patient discomfort. Instead of a static ramp that increases stimulation amplitude uniformly to the new target, various embodiments accelerate stimulation changes in regions where stimulation change is largely imperceptible to the patient (or does not cause discomfort) and then slow the stimulation changes as the stimulation approaches a therapeutic target or side effect. The ramped parameter may be amplitude, a pulse width, a pulse frequency, or fractionalization values for at least two electrodes. The ramped parameter may be a composite parameter that provides an indicator of delivered charge or an indicator of delivered dose based at least in part on values for at least two of an amplitude, a pulse width or a pulse frequency. Such composite parameters may address the situation where stimulation effects may more quickly change with smaller changes in amplitude when the pulses have larger pulse widths. The testing process may involve ramping values for more than one parameter.

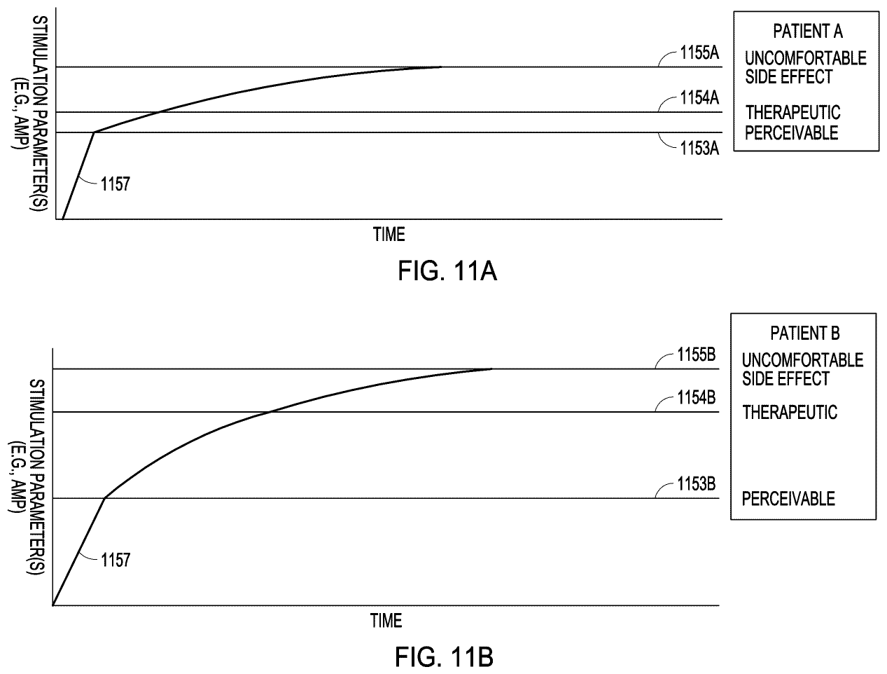

FIGS. 11A-11B illustrate, by way of example and not limitation, the use of a variable ramping protocols for two patients who experience some stimulation effects differently. Similar to FIGS. 10A-10B, the first patient may experience perceivable stimulation 1153A, therapeutic stimulation 1154A and uncomfortable stimulation 1155A at three different amplitudes that are relatively close together as illustrated in FIG. 11A, and the second patient may experience perceivable stimulation 1153B, therapeutic stimulation 1154B and uncomfortable stimulation 1155B differently at amplitudes that are relatively far apart as illustrated in FIG. 11B. In both of these examples of ramping protocols 1157, the stimulation may increase quickly until the perception threshold. Then the rate of increase may become smaller as the stimulation amplitude approaches the therapeutic threshold and the side effect threshold. This allows the therapy to be increased more gradually at these later levels and provide more time for the patient to respond and identify desired stimulation effects for the therapeutic threshold and/or undesired effects for the side effect threshold.

Generally, the ramping protocol may involve determining where the patient is expected to have a perception of a good or bad change. For example, using amplitude as an example of an adjustable parameter in a testing process, there is a certain amount of amplitude change that is likely imperceptible. The patient will experience a feeling of change at the perception threshold. That change may be a good stimulation effect. The desired therapeutic effect may be reached at a higher amplitude. However, at some point, change is going to become negative because of discomfort or other side effects. That is, by way of example and not limitation, there may be different regions of stimulation such as a region where changes in the parameter is not perceptible, another region where changes are perceptible and well-received by the patient, and another region where changes are uncomfortable or otherwise not well-received by the patient.

Various embodiments may implement a testing process with a boundary or more than one boundary where a stimulation effect is expected. The one or more boundaries may function as thresholds for changing the rate of change of the increase in a parameter value. For example, assuming that the time interval for testing a different parameter, such as amplitude, remains fixed, the parameter change interval (e.g., difference in amplitude) between adjacent tested points may change when the boundary is reached or is expected to be reached. Thus, for example, the parameter change interval may change to a smaller interval upon reaching the perception level, and the parameter change interval may become smaller as it is expected to reach the other stimulation effect boundaries such as the therapeutic threshold and side effect threshold. The ramping may typically progress to smaller parameter change intervals. In some embodiments, the ramping may progress with larger parameter change intervals. In some embodiments, the testing process may sometimes decrease the parameter change interval, such as when a boundary is expected, and then afterwards may sometimes increase the parameter change interval after the boundary when another boundary is not expected, and then decrease the parameter change interval again as another boundary is expected.

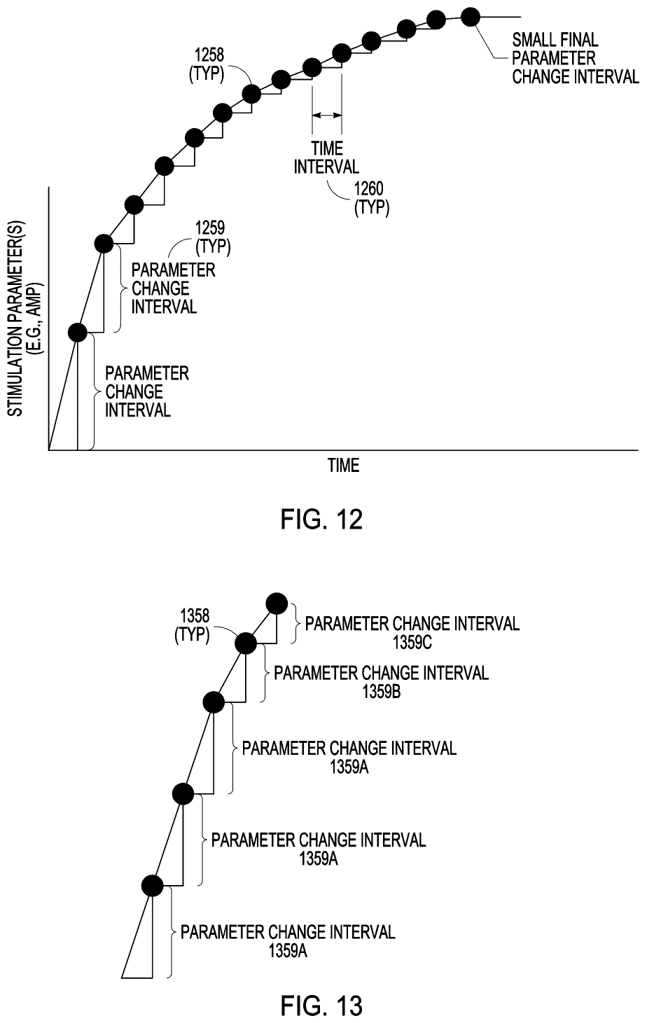

FIG. 12 illustrates an example of a ramping protocol for testing different parameter values in a parameter set. Each dot may represent a tested parameter value 1258 such as an amplitude value. The value-to-value difference 1259 between adjacent tested parameter values may vary. For example, when it is anticipated that the stimulation is approaching a side effect, the parameter change interval may decrease, which gives the patient more time to determine whether a side effect is being experienced and report that the side effect is being experienced. The time intervals 1260 between adjacent tests may be constant as illustrated, or may vary. For example, when it is anticipated that the stimulation is approaching a side effect, the time interval may increase to provide more time for the stimulation to wash in to confirm whether the stimulation causes a side effect.

FIG. 13 illustrates, by way of example and not limitation, a ramping protocol. Each dot may represent a tested parameter value 1358 such as an amplitude value. The illustrated example illustrates that a parameter change interval 1359A may stay the same for two or more tests or that a parameter change interval may 1359A, 1359B, 1359 C change for consecutive tests. Automatically progressing through the sequence of values may include using a same parameter change interval to progress through at least two consecutive values in the sequence of values and subsequently reducing the parameter change interval before progressing through a subsequent value in the sequence of values. Automatically progressing through the sequence of values may include using at least three different parameter change intervals to progress through three consecutive values in the sequence of values. The ramping protocol may be a pre-programmed protocol where each parameter change is predefined at predefined times. The ramping protocol may be based on a various inputs, a parameter change interval is based at least in part on one or more inputs. Some embodiments may generally follow a decreasing parameter change interval, but may have some parts of the sequence where the parameter change interval temporarily increases. By way of example and not limitation, the parameter change interval may decrease until the patient perceives the stimulation, and then temporarily increase the parameter change interval and then decrease the parameter change interval when the side effect is expected.

Various embodiments provide boundaries used to determine different parameter change rates. The rate of a parameter change may be reduced using smaller parameter change intervals and/or using longer periods of time before changing the parameter value by a parameter change interval. The rate of a parameter change may be increased using larger parameter change intervals and/or using shorter periods of time before changing the parameter value by a parameter change interval. The time being stimulation tests may be constant or may be variable. It is desirable to give a stimulation change time to wash in. Pauses or time intervals between tests before testing the next value (e.g., amplitude). The pauses or time intervals may be on an order of 15 or 30 seconds. Longer pauses may be provided as a probability of a stimulation event (e.g., side effect) increases. Some embodiments may preset the pauses by default. Some embodiments may enable the clinician to change the pauses or time intervals between tests. The system may be configured that, if feedback is received that the patient is experiencing a side effect, the parameter may be immediately returned to a value that did not cause the side effect.

Many of the examples provided above relate to DBS programming. The present subject matter may also be used in SCS therapy. Inputs, like medical imaging and/or sensing may be used to determine where the electrodes are to the spinal cord or nerve roots, which may be used to estimate the stimulation effects.

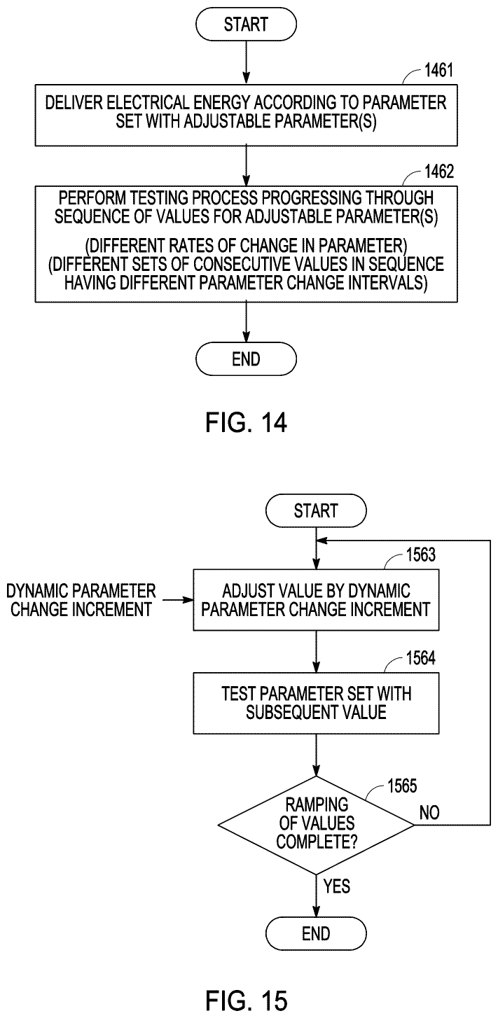

FIG. 14 illustrates, by way of example and not limitation, a method for testing parameter values for an adjustable parameter in a parameter set. The subject matter may include delivering electrical energy according to a stimulation parameter set. 1461 The electrical energy may be delivered using a neurostimulator. The stimulation parameter set may include at least one adjustable parameter such as, but not limited to, amplitude. The adjustable parameter(s) may include at least one of: an amplitude; a pulse width; a pulse frequency; fractionalization values for at least two electrodes; or a composite parameter. The composite parameter may provide an indicator of delivered charge or an indicator of delivered dose based at least in part on values for at least two of an amplitude, a pulse width or a pulse frequency. The subject matter may further include using a processing system to perform a testing process to test delivering electrical energy by automatically progressing through a sequence of values for the at least one adjustable parameter in the parameter set. 1462. There may be different rates of change in the parameter set. Different sets of consecutive values in a sequence may have different parameter change intervals. For example, a first value and a second value in the sequence of values may be separated by an initial parameter change interval. A second to last value and the last value in the sequence of values may be separated by a final parameter change interval. Progressing through the sequence of values may include reducing the parameter change interval from the initial parameter change interval to the final parameter change interval. The timing for delivering electrical energy using a current one of the sequence of values before moving to a subsequent one of the sequence of values may be longer than a wash-in time for the electrical therapy to cause a neural stimulation effect.

The testing process may be pre-programmed to test a specific sequence of values with the parameter change interval progressively reduced from the initial parameter change interval to the final parameter change interval. Each value may be a pre-programmed value. In some embodiments, the parameter change interval may depend on the at least one received input. By way of example, a user input may include data indicative of a spatial relationship between a representation of an anatomical structure and a stimulation field model representing a volume of tissue activated by the delivered electrical energy. The parameter change interval may depend on the spatial relationship. The input may include recorded patient data of a stimulation effect for a specific stimulation parameter set. The recorded patient data may be used to estimate a stimulation effect probability for other stimulation parameter sets and the parameter change interval may depend on the estimated stimulation effect probability. The input may include user data indicative of a user's programming preference and the parameter change interval may depend on the user's programming preference. The input may include aggregate data collected from multiple patients, which may be used to estimate a stimulation effect probability for other stimulation parameter sets. The parameter change interval may depend on the estimated stimulation effect probability. The input may include an estimated change in stimulation effect for the delivered electrical energy between adjacent steps in the sequence of values and the parameter change interval may depend on the estimated change. The input may include a manual input indicative of at least one transition point in the sequence of values for transitioning to a different parameter change interval and the parameter change interval may depend on the at least one transition point. For example, the transition point(s) may correspond to a perception threshold and/or side effect threshold.

FIG. 15 illustrates, by way of example and not limitation, a testing process. The testing process may be a specific embodiment of the testing process performed at 1462 in FIG. 14. The testing process may adjust a value for a parameter by a dynamic parameter change increment as illustrated at 1563. The dynamic parameter change increment may be based on a preprogrammed values or may be based on one or more input(s). The adjusted value provides a subsequent value for the parameter, and the parameter set may be tested with this subsequent value 1564. At 1565, it is determined whether the ramping of values is complete. If not complete, the process may proceed to 1563 to adjust the value. If the ramping is complete, the testing process may terminate or may proceed to test a different parameter set which may or may not include a different adjustable parameter.

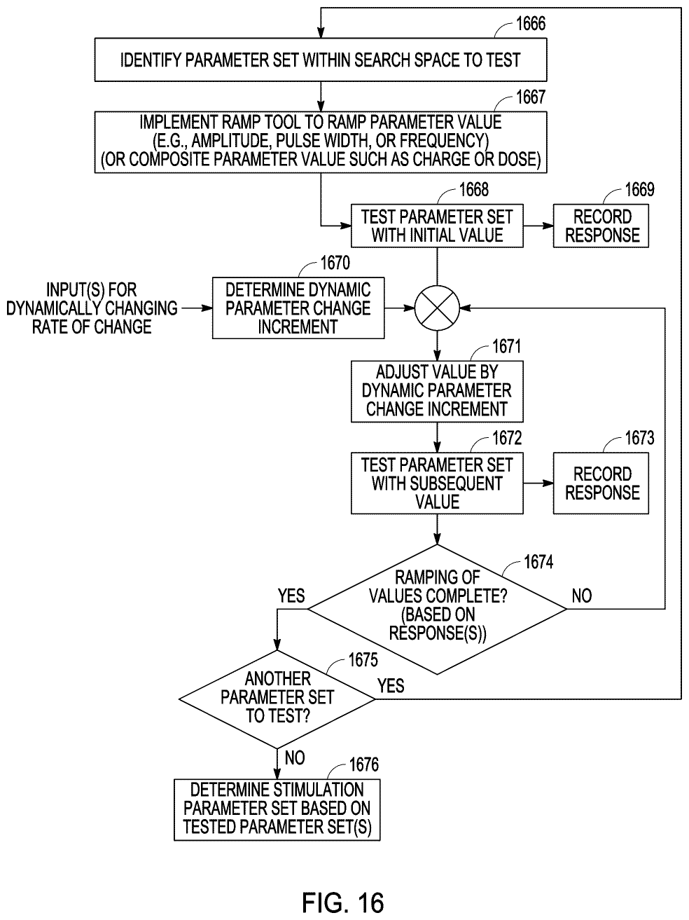

FIG. 16 illustrates, by way of example and not limitation, a more detailed example of a testing process that includes determining a dynamic parameter change increment used to adjust a value of the adjustable parameter in the parameter set. The testing process may be a specific embodiment of the testing process performed at 1462 in FIG. 14. At 1666, the process may include identifying a parameter set within a search space to test. At 1667, a ramp tool may be implemented to ramp a parameter value. The ramped value may include, by way of example or not limitation, an amplitude, a pulse width, a frequency, or composite value(s) such as charge or stimulation dose which may include a composite of at least amplitude and pulse width. At 1668, the parameter set is tested with the adjustable parameter value set to an initial value. The response may be recorded at 1669. A dynamic parameter change increment may be determined and used to adjust a value of the adjustable parameter in the parameter set 1670. The dynamic parameter change increment may be based on a preprogrammed values or may be based on one or more input(s). The adjusted value may be used to provide a subsequent value for the parameter 1671, and the parameter set may be tested with this subsequent value 1672. The response may be recorded at 1673. At 1674, it is determined whether the ramping of values is complete. If not complete, the process may proceed to adjust the value by the dynamic parameter change increment 1670, 1671. If the ramping is complete, the testing process may terminate or may proceed to test a different parameter set 1675 and return to 1666. If there is not another parameter set to test, the process may proceed to determine the stimulation parameter set based on the tested parameter set(s) 1676.

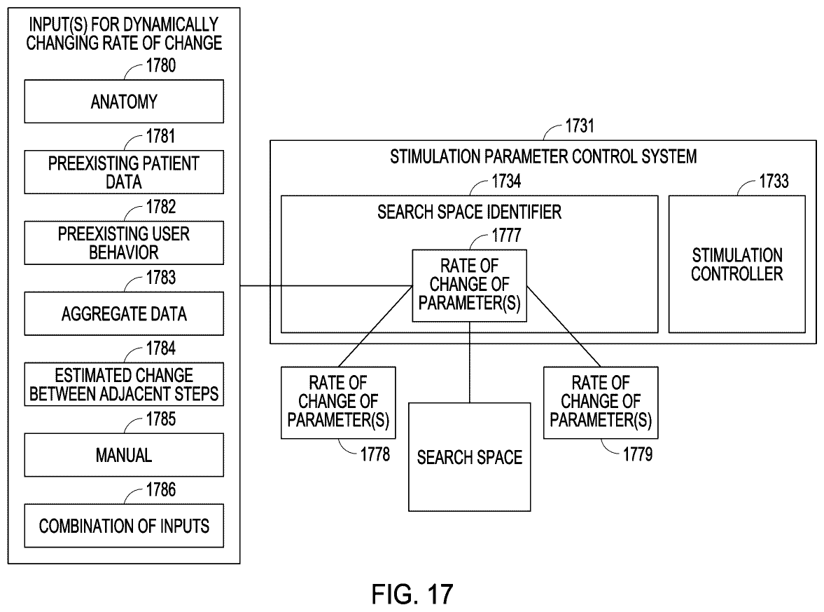

FIG. 17 illustrates, by way of example and not limitation, a system for providing a variable parameter value interval to test parameter values within a stimulation parameter set. The system may include a neurostimulator configured to deliver electrical energy according to a stimulation parameter set. The stimulation parameter set may include at least one adjustable parameter. The illustrated system may be an embodiment of the system of FIG. 5, and may include a stimulation parameter control system 1731 which may include a stimulator controller 1733 used to control the stimulator. The stimulation parameter control system 1731 may include a search space identifier 1734 used to identify a portion of the search space to test. The search space identifier may be configured to control a rate of change of parameter(s) 1777. The rate of change may be dynamically changed using one or a combination of two or more inputs. The search space identifier 1734 may also be configured to receive a sensitive patient indicator 1778 and/or a user-inputted override 1779.

Some embodiments may set a boundary using anatomy 1780. For example, as a stimulation field model (SFM) of stimulation approaches a known or manually identified side effect structure, the rate of parameter change may slow down such that there are more intervals before the side effect structure is overlapped. The user may estimate the side effect based on what the user is seeing in the patient's anatomy, how the lead is positioned, what anatomical structures are closer at certain positions than others. Imaging may provide position information where the electrodes are to the spine, how lateral or midline the electrodes are, what a potential side effect may be, and the like. This information may be used to determine an estimated boundary for the values of the adjustable parameter(s) based on the anatomy.

Some embodiments may set a boundary using preexisting patient data 1781. Recorded preexisting patient data may indicate at least one side effect at a given stimulation setting, which then can be used to estimate a probability of side effects for other stimulation settings (e.g., for other portions of the lead). Patient data from other stimulation locations that have been tested can be used to estimate where stimulation effect threshold(s) may be for other locations that have not yet been tested. This patient data may include where you previously recorded side effects, where your previously recorded benefits, and where you previously recorded that the stimulation was not perceptible.

Some embodiments may set a boundary using preexisting user behavior 1782. Clinicians may have individual approaches or preferences to programming. For example, physicians may not increase stimulation beyond a specific upper limit. This limit may be used as a boundary. Clinicians may also increase stimulation amplitudes rapidly at low amplitudes, then increase stimulation amplitudes slower, and then increase stimulation amplitude even lower as an upper limit or an anatomical target is reached. As stimulation approaches this limit, the rate of parameter change could be slowed. Slowing the rate of parameter change may include reducing the parameter change interval and/or using longer periods of time before changing the parameter value by a parameter change interval.

Some embodiments may set a boundary using aggregate data 1783 collected from multiple patients which may include the current patient. A system may assume common relationships based on data collected from multiple patients particularly for anatomy and for general trends in side effect location relative to other known side effects.

Some embodiments may set a boundary change using an estimated change in a stimulation effect between adjacent steps 1784. For example, rates of parameter change may start at lower rates when the stimulation effect for two adjacent steps of change are very far apart. Thus, change from one to the other constitutes a large change in stimulation. For example, the stimulation effect from stimulation using pulses with larger pulse widths may change faster with increasing amplitude than the stimulation effect using pulses with smaller pulse widths. For a given pulse amplitude, the larger pulse widths deliver more charge (or more charge per unit of time that includes multiple pulses) than smaller pulse widths. In another example, the stimulation effect may quickly increase when a stimulated region jumping between anatomical regions with a large difference in impedance. In another example, the stimulation effect may quickly increase when there is a large difference in amplitudes that were previously tested.

Some embodiments may set a boundary change using a manual input from the clinician 1785. For example, a programming clinician may set individual thresholds at or before which the rate of parameter change slows down.

The above-identified boundaries may be used individually or two or more of these boundaries may be used in conjunction with one another 1786. For example, some embodiments may set a boundary using both anatomy and preexisting patient data. The boundaries may be weighted. For example, the patient data may be weighted higher than the anatomy when setting a boundary.

The estimated thresholds or boundaries may be estimates based on probabilities of the stimulation effect occurring. The rate of parameter changes may be set to address that which is most likely to occur. For example, if the probability of side effects is estimated to be less than 20%, the rate of parameter change may be set to be at a first rate; and if the probability of side effects is later estimated to be 80% with the increasing parameter value (e.g., amplitude), the rate of parameter change may be set to a second rate lower than the first rate.

However, some patients will be outliers, and it is desirable to be able to accommodate their need to balance avoidance of discomfort while still providing a quick programming process. For example, some patients are more sensitive to stimulation. Some embodiments may enable a user may tag that the patient is sensitive, which may automatically reduce the rate of parameter change normally provided by the testing process. The reduction may be automatically implemented or may be entered by the clinician. For patients highly sensitive to stimulation, stimulation is felt even at the lowest thresholds and sudden changes can be uncomfortable. Patients can be tagged as being “sensitive” to stimulation 1778. The sensitive tag may override other speed recommendations and slow down all stimulation changes. Some patients may only be sensitive to stimulation changes above set threshold or when stimulation has specific properties (rates, pulse width, regions of the lead, etc.). The tag mentioned above could be associated with specific parameters or thresholds for a given patient. By way of example and not limitation, the patient may respond normally when the stimulation amplitude is below 4 mA. However, above 4 mA, the patient response is more sensitive compared to other patients. The rate of parameter change may slow down more above 4 mA than for non-sensitive patients. More time may be needed for the comfort of sensitive patients than for non-sensitive patients.

Some embodiments allow a user to input an override command 1779 that overrides the various input processing system from progressively reducing the parameter change interval. The override may be a pause or stop (and restart) and ongoing ramp, or may include increasing or decreasing a rate of a parameter change. For example, actuating a fast forward button one or more times may increase the rate of parameter change. The system may be configured to provide a warning if the user is attempting to change the parameter value to something that is likely to be very uncomfortable for the patient.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

Method examples described herein may be machine or computer-implemented at least in part. Some examples may include a computer-readable medium or machine-readable medium encrypted with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media (referred to herein as computer readable medium), such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like. The term “machine” may include at least one processor/controller, including one processor/controller to implement all of the instructions, at least two processors/controllers where one processor/controller operates on some of the instructions and other processor(s)/controller(s) operate on other instructions, or at least two processors/controllers where each processor/controller is capable of operating on the same instructions. Thus, for example, distributed systems or systems with shared resources are contemplated.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A method, comprising:
delivering electrical energy according to a stimulation parameter set using a neurostimulator, wherein the stimulation parameter set includes at least one adjustable parameter; and
using a processing system to perform a testing process to test delivering electrical energy by automatically progressing through a sequence of values for the at least one adjustable parameter in the parameter set, wherein a first value and a second value in the sequence of values are separated by an initial parameter change interval and a second to last value and the last value in the sequence of values are separated by a final parameter change interval, and wherein the progressing through the sequence of values includes reducing the parameter change interval from the initial parameter change interval to the final parameter change interval.

2. The method of claim 1, wherein the at least one adjustable parameter includes at least one of:
an amplitude;
a pulse width;
a pulse frequency;
fractionalization values for at least two electrodes; or
a composite parameter, wherein the composite parameter provides an indicator of delivered charge or an indicator of delivered dose based at least in part on values for at least two of an amplitude, a pulse width or a pulse frequency.

3. The method of claim 1, wherein the automatically progressing through the sequence of values includes:
using a same parameter change interval to progress through at least two consecutive values in the sequence of values and subsequently reducing the parameter change interval before progressing through a subsequent value in the sequence of values; or
using at least three different parameter change intervals to progress through three consecutive values in the sequence of values.

4. The method of claim 1, wherein timing for delivering electrical energy using a current one of the sequence of values before moving to a subsequent one of the sequence of values is longer than a wash-in time for the electrical energy to cause a neural stimulation effect.

5. The method of claim 1, wherein the testing process is pre-programmed to test a specific sequence of values with the parameter change interval progressively reduced from the initial parameter change interval to the final parameter change interval.

6. The method of claim 1, further comprising receiving at least one input, wherein the parameter change interval depends on the at least one received input.

7. The method of claim 6, wherein the at least one input includes data indicative of a spatial relationship between a representation of an anatomical structure and a stimulation field model representing a volume of tissue activated by the delivered electrical energy, and the parameter change interval depends on the spatial relationship.

8. The method of claim 6, wherein the at least one input includes recorded patient data of a stimulation effect for a specific stimulation parameter set, the method includes estimating a stimulation effect probability for other stimulation parameter sets using the recorded patient data, and the parameter change interval depends on the estimated stimulation effect probability.

9. The method of claim 6, wherein the at least one input includes user data indicative of a user's programming preference, and the parameter change interval depends on the user's programming preference.

10. The method of claim 6, wherein the at least one input includes aggregate data collected from multiple patients, the method includes estimating a stimulation effect probability for other stimulation parameter sets using the aggregate data, and the parameter change interval depends on the estimated stimulation effect probability.

11. The method of claim 6, wherein the at least one input includes an estimated change in stimulation effect for the delivered electrical energy between adjacent steps in the sequence of values, and the parameter change interval depends on the estimated change.

12. The method of claim 6, wherein the at least one input includes a manual input indicative of at least one transition point in the sequence of values for transitioning to a different parameter change interval, and the parameter change interval depends on the at least one transition point.

13. The method of claim 1, wherein:
the at least one input includes at least two inputs selected from:
data indicative of a spatial relationship between a representation of an anatomical structure and a stimulation field model representing a volume of tissue activated by the delivered electrical energy;
recorded patient data of a stimulation effect for a specific stimulation parameter set, and the method includes estimating a stimulation effect probability for other stimulation parameter sets using the recorded patient data;
user data indicative of a user's programming preference;
aggregate data collected from multiple patients, and the method includes estimating a stimulation effect probability for other stimulation parameter sets using the aggregate data;
an estimated change in stimulation effect for the delivered electrical energy between adjacent steps in the sequence of values; or
a manual input indicative of at least one transition point in the sequence of values for transitioning to a different parameter change interval, and
wherein the parameter change interval depends on the at least two inputs.

14. The method of claim 1, further comprising receiving a sensitive patient indicator, and the parameter change interval depends on the sensitive patient indicator.

15. The method of claim 1, further comprising receiving a user-inputted override for overriding the processing system from progressively reducing the parameter change interval when automatically progressing through the sequence of values for the at least one adjustable parameter in the parameter set.

16. A non-transitory machine-readable medium including instructions, which when executed by a machine, cause the machine to perform a method, comprising:
delivering electrical energy according to a stimulation parameter set using a neurostimulator, wherein the stimulation parameter set includes at least one adjustable parameter; and
using a processing system to perform a testing process to test delivering electrical energy by automatically progressing through a sequence of values for the at least one adjustable parameter in the parameter set, wherein a first value and a second value in the sequence of values are separated by an initial parameter change interval and a second to last value and the last value in the sequence of values are separated by a final parameter change interval, and wherein the progressing through the sequence of values includes reducing the parameter change interval from the initial parameter change interval to the final parameter change interval.

17. A system, comprising:
a neurostimulator configured to deliver electrical energy according to a stimulation parameter set, wherein the stimulation parameter set includes at least one adjustable parameter;
a processing system configured to perform a testing process to test delivering electrical energy by automatically progressing through a sequence of values for the at least one adjustable parameter in the parameter set, wherein a first value and a second value in the sequence of values are separated by an initial parameter change interval and a second to last value and the last value in the sequence of values are separated by a final parameter change interval, and wherein the progressing through the sequence of values includes reducing the parameter change interval from the initial parameter change interval to the final parameter change interval.

18. The system of claim 17, wherein the automatically progressing through the sequence of values includes using a same parameter change interval to progress through at least two consecutive values in the sequence of values and subsequently reducing the parameter change interval before progressing through a subsequent value in the sequence of values.

19. The system of claim 17, wherein the automatically progressing through the sequence of values includes using at least three different parameter change intervals to progress through three consecutive values in the sequence of values.

20. The system of claim 17, wherein timing for delivering electrical energy using a current one of the sequence of values before moving to a subsequent one of the sequence of values is longer than a wash-in time for the electrical energy to cause a neural stimulation effect.
